(19) United States
(12) Patent Application Publication
(10) Pub. No.: US 2025/0360205 A1
(43) Pub. Date: Nov. 27, 2025

(54) NOD1 MODULATORS AND USES THEREOF

(71) Applicant: Academia Sinica, Taipei (TW)

(72) Inventors: Wei-Chieh CHENG, Taipei City (TW); Hung-Chang TSAI, Pingtung County (TW)

(21) Appl. No.: 18/872,315

(22) PCT Filed: Jun. 9, 2023
(86) PCT No.: PCT/US2023/024893

§ 371 Date: Dec. 6, 2024

Related U.S. Application Data
(60) Provisional application No. 63/350,850, filed on Jun. 9, 2022.

(51) Int. Cl. A61K 39/39; A61K 39/00; A61K 39/145; A61P 37/04; C07K 5/083

(52) U.S. Cl. CPC A61K 39/39; A61K 39/145; A61P 37/04; C07K 5/0806; A61K 2039/55516; A61K 2039/575; C12N 2760/16134

(57) ABSTRACT

Disclosed herein are compounds having adjuvant properties, vaccines including the adjuvant compounds, and their uses in the prophylaxis or therapy for respiratory virus infection.

### CROSS REFERENCES TO RELATED APPLICATION

This application claims priority and the benefit of U.S. Provisional Patent Application No. 63/350,850, filed Jun. 9, 2022, the entirety of which is incorporated herein by reference.

### BACKGROUND OF THE INVENTION

### 1. FIELD OF THE INVENTION

The present disclosure in general relates to compounds having adjuvant properties, vaccines comprising the adjuvant compounds, and their uses in prophylaxis or therapy for respiratory virus infection.

### 2. DESCRIPTION OF RELATED ART

Bacterial peptidoglycans (PGNs) and their fragments are a novel class of pathogen-associated molecular patterns (PAMPs), and are explicitly recognized by various host pattern recognition receptors (PRRs) including nucleotide-binding oligomerization domain-like receptors (NOD-like receptors), a family of peptidoglycan recognition proteins (PGRPs), and C-type lectin receptors (CLRs), leading to the induction of host innate immune responses. Further, these agonists can be applied in the adjuvant research.

It was known that PGN fragments incorporating the unique amino acid (2S, 6R)-2,6-diaminopimelic acid (meso-DAP), such as γ-D-Glu-meso-DAP (iE-meso-DAP or iE-DAP) or L-Ala-γ-D-Glu-meso-DAP (A-iE-meso-DAP or A-iE-DAP) stimulate cytosolic NOD1 activity leading to activation of the NF-κB pathway, the production of inflammatory cytokines, and host autophagy. The inventors of the present disclosure synthesized novel chemically modified PGN fragments, which were further confirmed to be NOD1 agonists with adjuvant properties. Thus, these newly identified NOD1 agonists are useful as adjuvants for enhancing the immune response elicited by an immunogen (e.g., a respiratory virus or a fragment thereof).

### SUMMARY

Inventors of the present disclosure designed and synthesized novel NOD1 agonists inspired by the bacterial peptidoglycan-based tripeptide, L-Ala-γ-D-Glu-mDAP (A-iE-meso-DAP), and synthesized using ionic liquid supported chemistry and the orthogonally protected (2R, 6S)-2,6-diamino-7-hydroxyheptanoic acid (OP-DAHH). These newly produced NOD1 agonists are found to possess adjuvant properties, thus are useful in the prophylaxis or therapy for influenza.

Accordingly, one aspect of the present disclosure is to provide a novel compound having the structure of formula (I), a pharmaceutically acceptable salt, a solvate or a stereoisomer thereof,

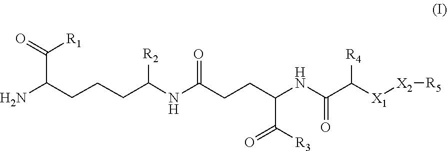

wherein,
X₁ and X₂ are independently nil, —NH or —(C═O);
R₁ and R₃ are independently hydroxyl or alkoxy;
R₂ is —COOH or —CH₂OH;
R₄ is hydrogen or C₁₋₆ alkyl;
R₅ is hydrogen or C₁₋₂₀ alkyl optionally substituted with a substituent selected from the group consisting of cycloalkyl, phenyl, biphenyl and —O-phenyl; and
the phenyl, the biphenyl or the —O-phenyl is optionally substituted with one or more halo, alkyl, or alkoxy.

According to some embodiments of the present disclosure, X₁ is nil, X₂ is —NH, R₁ is hydroxy, R₂ is methoxy, R₃ is hydroxy, R₄ is isopropyl or isobutyl, and R₅ is hydrogen. Exemplary compounds include, but are not limited to,

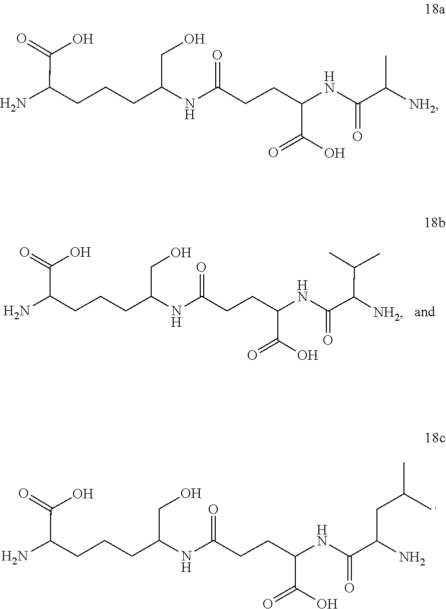 [SMILES: CC(N)C(=O)NC(CCC(=O)NC(CO)CCCC(N)C(=O)O)C(=O)O ; CC(C)C(N)C(=O)NC(CCC(=O)NC(CO)CCCC(N)C(=O)O)C(=O)O ; CC(C)CC(N)C(=O)NC(CCC(=O)NC(CO)CCCC(N)C(=O)O)C(=O)O]

According to some embodiments of the present disclosure, particular compounds are of formula (II),

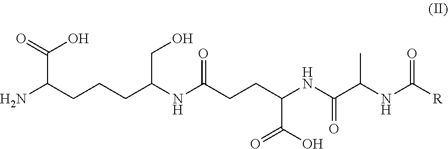

wherein,
R is C₁₋₂₀ alkyl optionally substituted with a substituent selected from the group consisting of cycloalkyl, phenyl, biphenyl and —O-phenyl; and
the phenyl, the biphenyl or the —O-phenyl is optionally substituted with one or more halo, alkyl, or alkoxyl.

According to some embodiments, the compound of formula (II) is selected from the group consisting of,

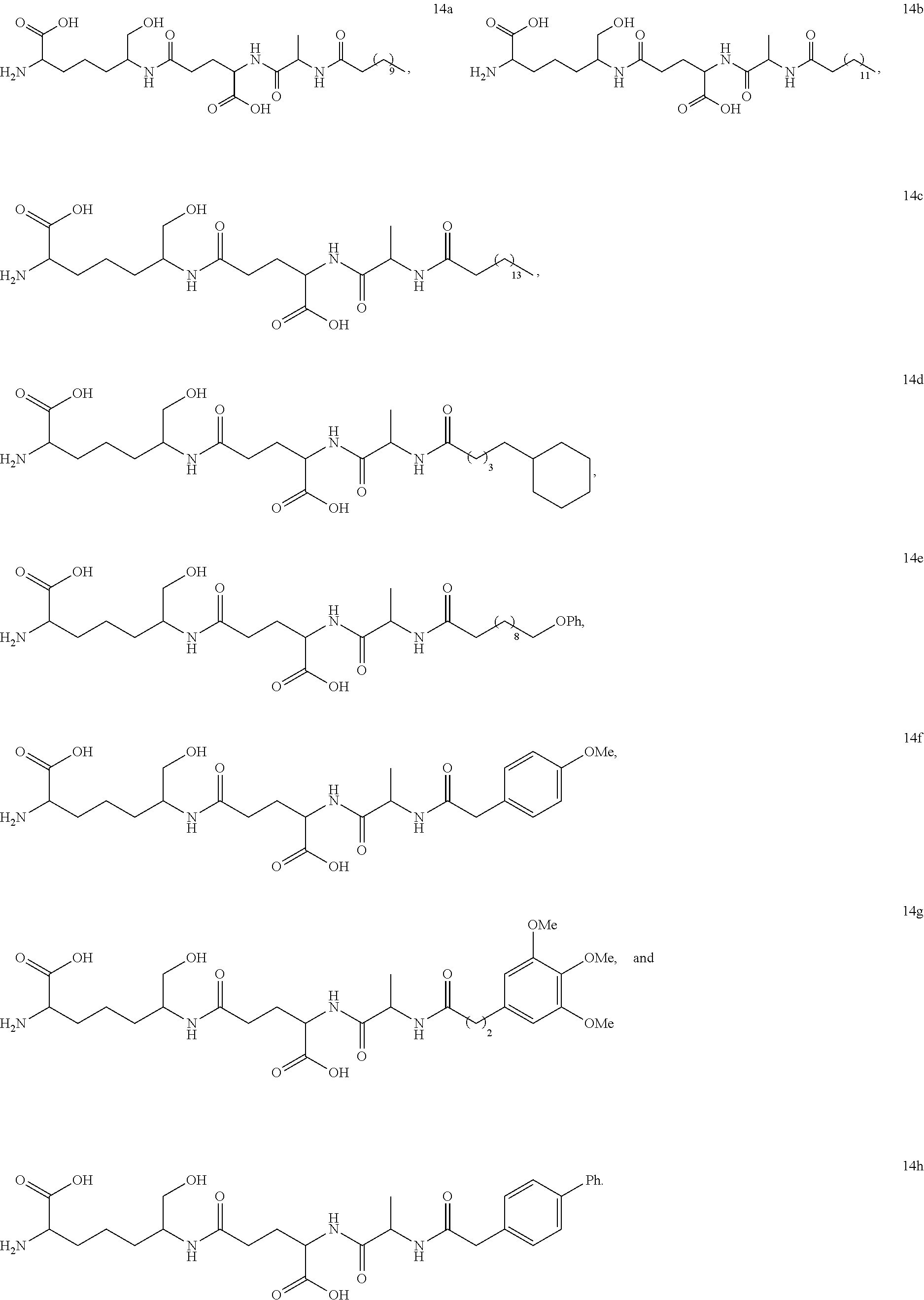

According to one preferred embodiment of the present disclosure, the compound of formula (II) is

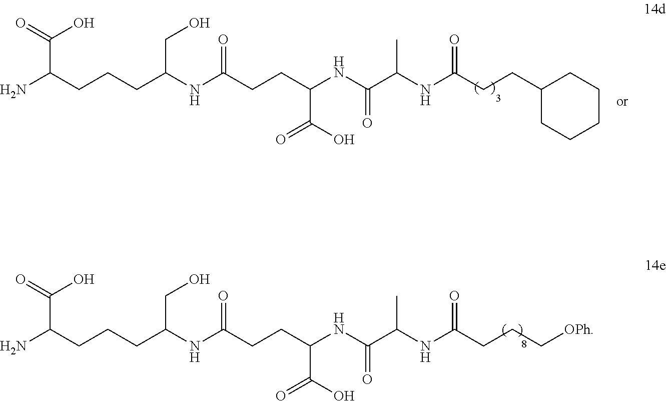

According to further embodiments of the present disclosure, particular compounds are of formula (III),

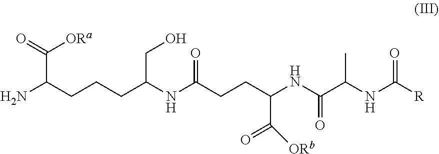

wherein,
R^{a} and R^{b} are independently C₁₋₆ alkyl;
R is C₁₋₂₀ alkyl optionally substituted with a substituent selected from the group consisting of cycloalkyl, phenyl, biphenyl and —O-phenyl; and
the phenyl, the biphenyl or the —O-phenyl is optionally substituted with one or more halo, alkyl, or alkoxyl

According to some embodiments, the compound of formula (III) is selected from the group consisting of,

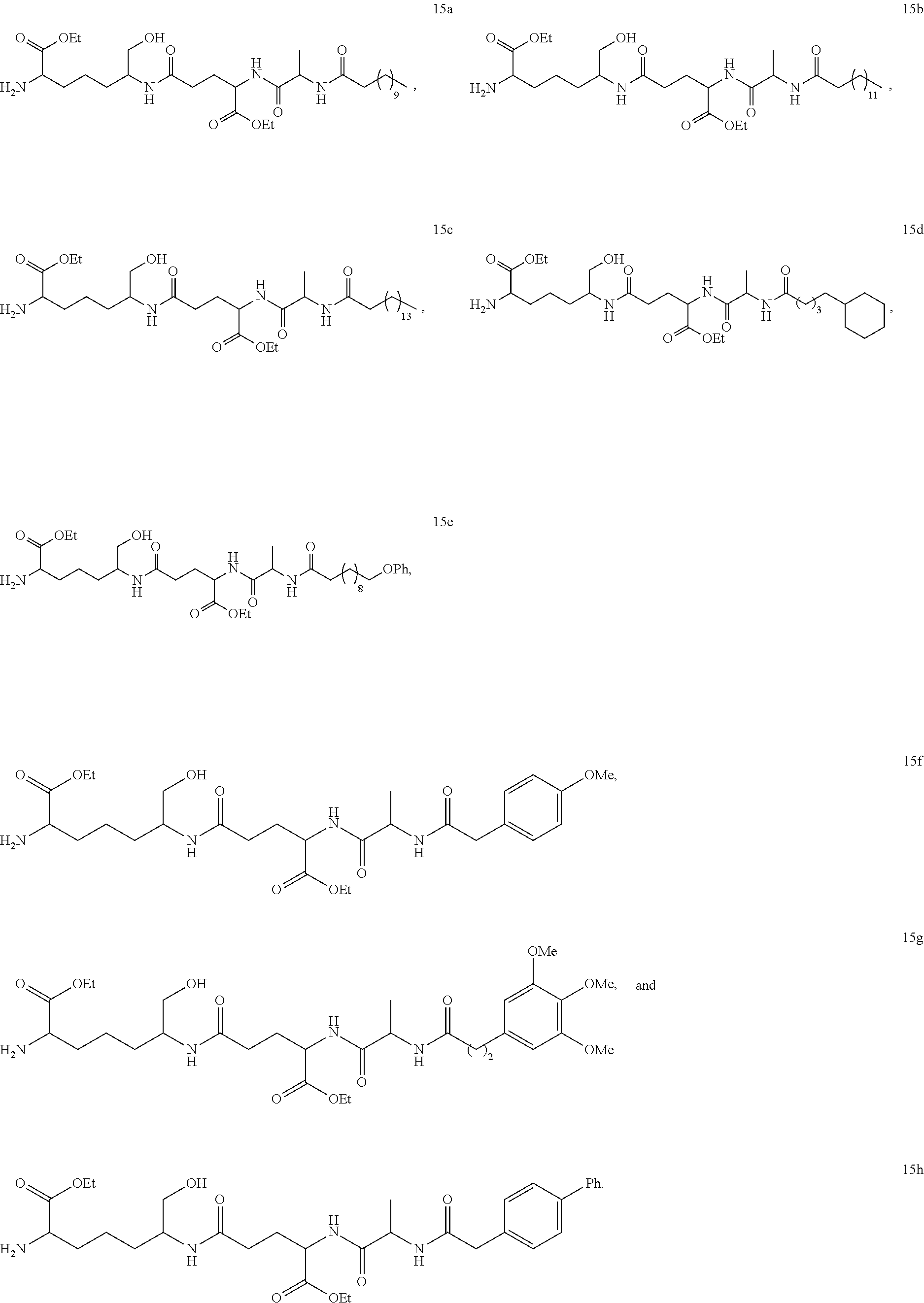

According to further embodiments of the present disclosure, particular compounds are of formula (IV),

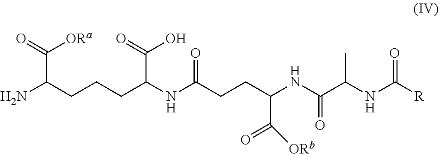

wherein,
R^{a} and R^{b} are independently C₁₋₆ alkyl;
R is C₁₋₂₀ alkyl optionally substituted with a substituent selected from the group consisting of cycloalkyl, phenyl, biphenyl and —O-phenyl; and
the phenyl, the biphenyl or the —O-phenyl is optionally substituted with one or more halo, alkyl, or alkoxyl.

According to some embodiments, the compound of formula (IV) is selected from the group consisting of.

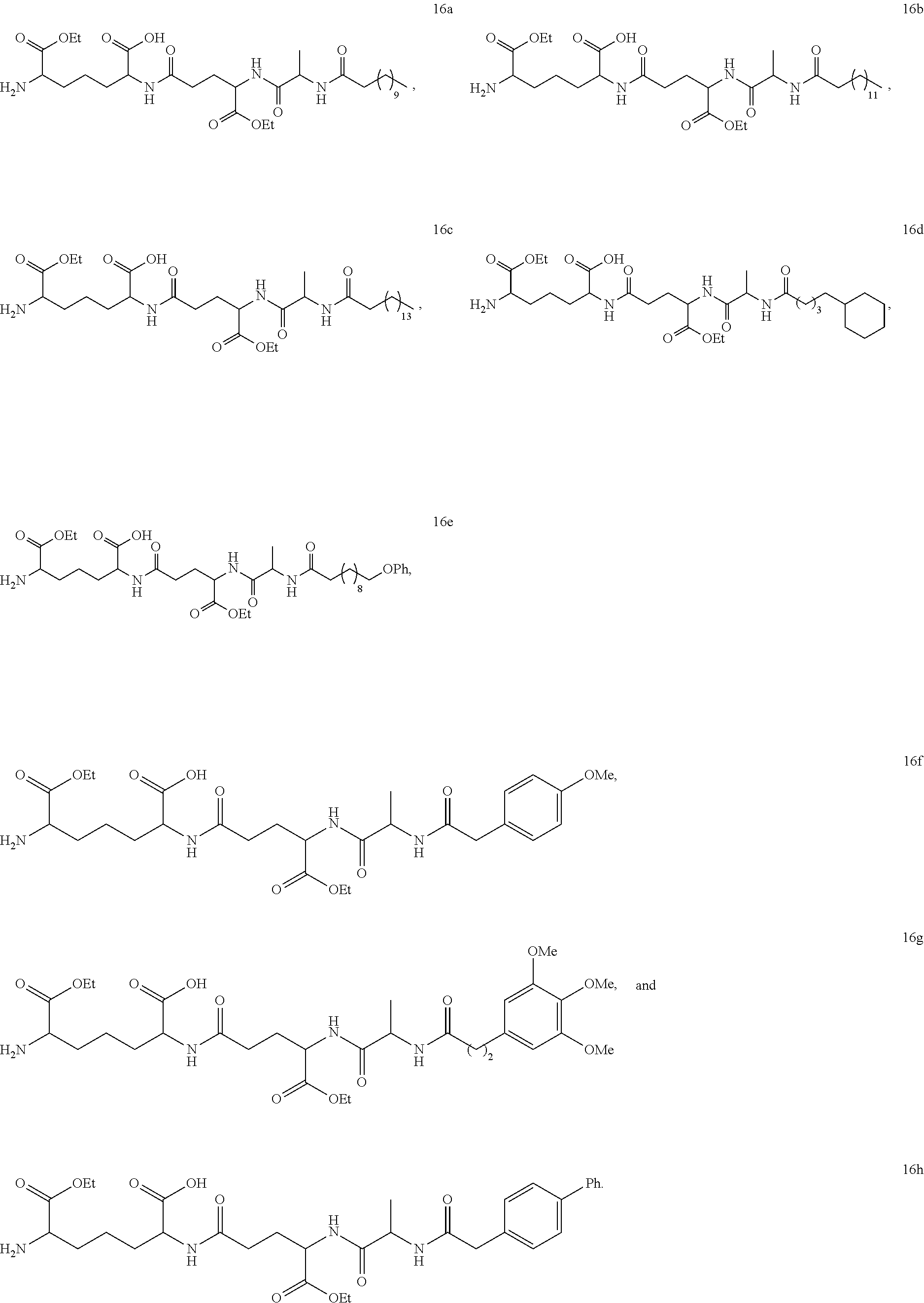

According to still further embodiments of the present disclosure, particular compounds are of formula (V),

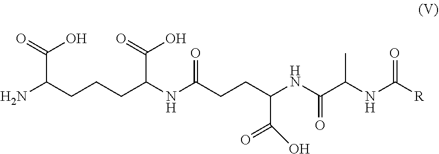

wherein,
R is C₁₋₂₀ alkyl optionally substituted with a substituent selected from the group consisting of cycloalkyl, phenyl, biphenyl and —O-phenyl; and
the phenyl, the biphenyl or the —O-phenyl is optionally substituted with one or more halo, alkyl, or alkoxyl.

According to some embodiments, the compound of formula (V) is selected from the group consisting of,

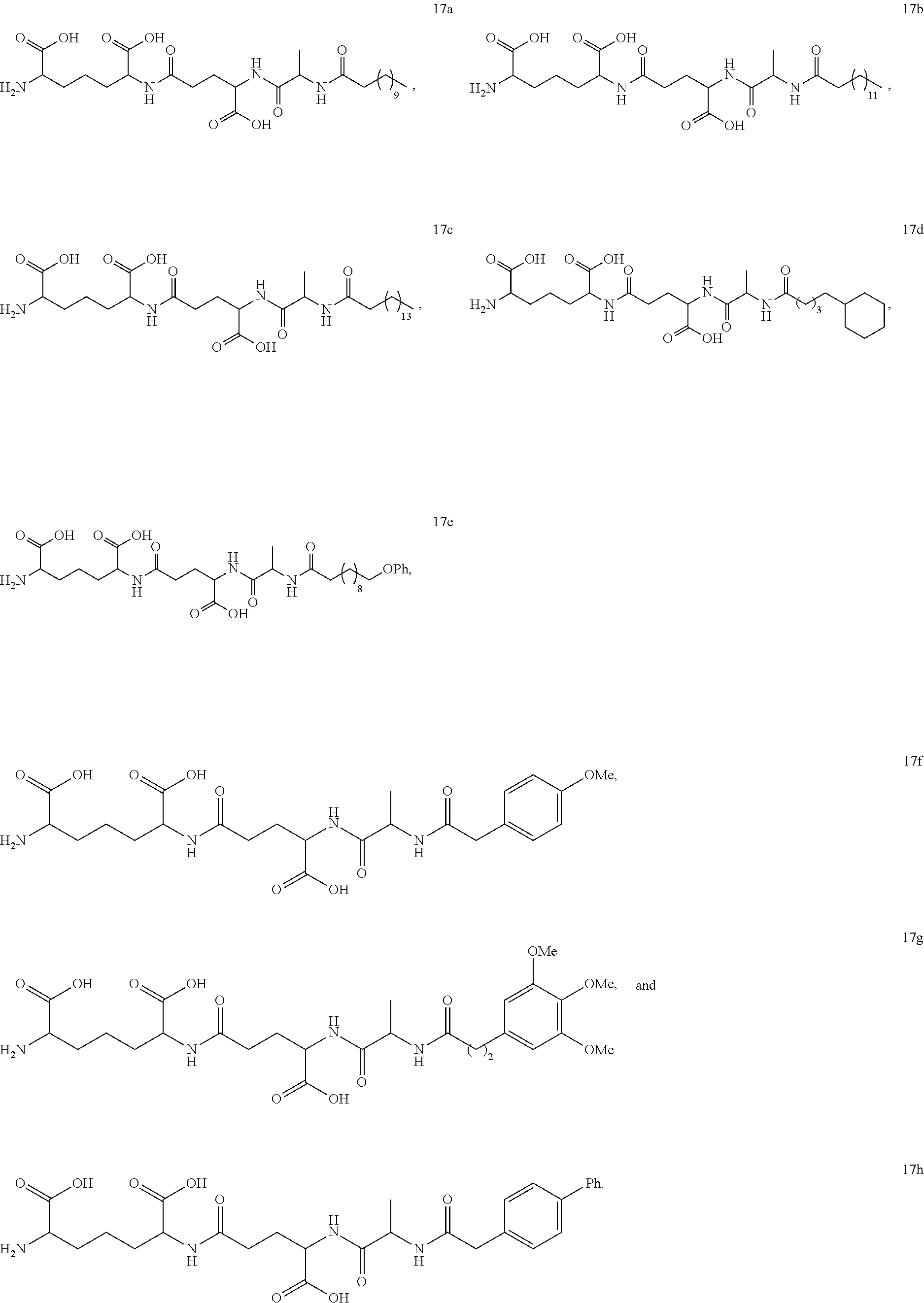

According to other embodiments of the present disclosure, particular compounds are of formula (VI),

wherein,

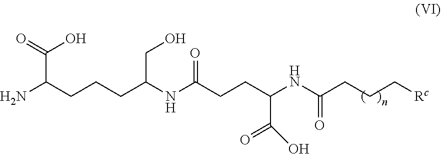

n is an integral between 3 to 10;
R^{c} is H, cycloalkyl, phenyl, biphenyl or —O-phenyl; and
the phenyl, the biphenyl or the —O-phenyl is optionally substituted with one or more halo, alkyl, or alkoxyl.

According to some embodiments, the compound of formula (VI) is selected from the group consisting of,

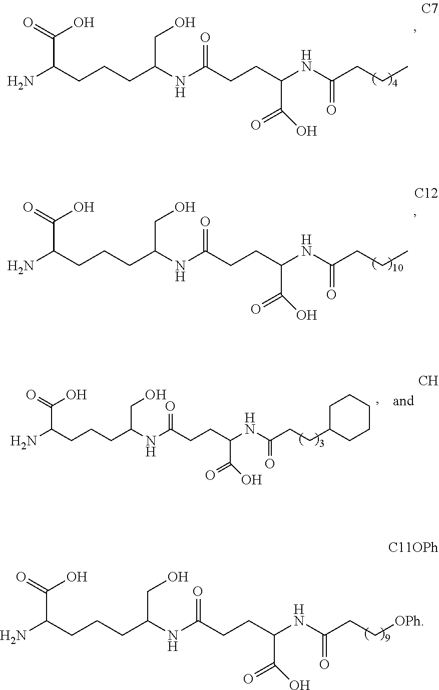

The second aspect of the present disclosure is to provide a vaccine, which comprises an immunogen and the compound of formula (I), its pharmaceutically acceptable salt, solvate or stereoisomer, wherein the immunogen is capable of eliciting an immune response against a respiratory virus, and the compound of formula (I), its pharmaceutically acceptable salt, solvate or stereoisomer is capable of enhancing the immune response elicited by the immunogen.

According to embodiments of the present disclosure, the immunogen is an antigen selected from the group consisting of a subunit antigen, an inactivated whole virus, a live attenuated virus, and a combination thereof.

According to preferred embodiments of the present disclosure, the compound of formula (I) is

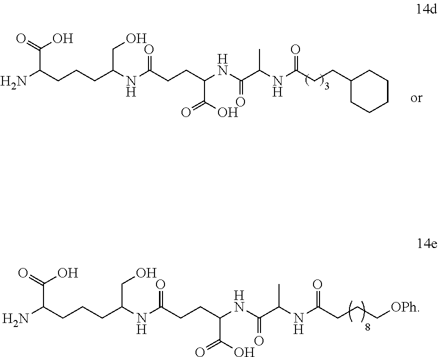

According to preferred embodiments of the present disclosure, the respiratory virus is an influenza virus, such as an avian influenza virus, or a seasonal influenza virus. Examples of the avian influenza virus include, but are not limited to, H5N1 and H7N9.

The third aspect of the present disclosure is to provide a method of immunizing a subject against a viral respiratory infection. The method includes administering to the subject an effective amount of the vaccine of the present disclosure.

According to embodiments of the present disclosure, the immunogen comprised in the vaccine is an antigen selected from the group consisting of a subunit antigen, an inactivated whole virus, a live attenuated virus, and a combination thereof.

According to preferred embodiments of the present disclosure, the compound of formula (I) comprised in the vaccine is

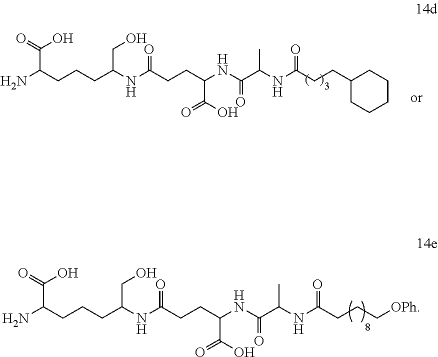

According to preferred embodiments of the present disclosure, the respiratory virus is an influenza virus, such as an avian influenza virus, or a seasonal influenza virus. Examples of the avian influenza virus include, but are not limited to, H5N1 and H7N9.

In all embodiments of the present disclosure, the subject is a human

The details of one or more embodiments of this disclosure are set forth in the accompanying description below. Other features and advantages of the invention will be apparent from the detail descriptions, and from claims.

### BRIEF DESCRIPTION OF THE DRAWINGS

The accompanying drawings, which are incorporated in and constitute a part of the specification, illustrate various example systems, methods and other exemplified embodiments of various aspects of the invention. The present description will be better understood from the following detailed description read in light of the accompanying drawings, where,

FIG. 1. NOD1 stimulation activity of N-substituted tripeptides 14-17. The NF-κB activity was measured in HEK-Blue hNOD1-specific cells after incubation of a candidate compound for 16 h at a concentration of 0.1 μM. The reference compounds are iE-DAP (Ref 1), C12-iE-DAP (Ref 2), A-iE-DAP (also called Tri-DAP, Ref 3). All experiments were performed in triplicate.

FIG. 2. NOD1 stimulation activity of selected tripeptides 14a, 14b, 14d, and 14e. The NF-κB activity was measured in HEK-Blue hNOD1-specific cells after incubation of the selected candidate for 16 h at 1 μM and 0.1 μM. C12-iE-DAP is used as a reference compound. All experiments were performed in triplicate.

FIG. 3. NOD1 stimulation activity of compounds 6-9. The NF-κB activity was measured in HEK-Blue hNOD1-specific cells after incubation of the selected candidate for 16 h at 1 μM and 0.1 μM. The reference compounds are iE-DAP. All experiments were performed in triplicate.

FIG. 4. The end-point ELISA titer and IFN-γ-secreting cells induced in mice immunized with HA adjuvanted with alumium oxide (alum), 14d, or 14e. Mice were immunized with antigen (Ag), or antigen adjuvanted with 8 or 80 ug of 14d and 14e. After two immunizations, the mice serum ware collected and analyzed for the antibodies targeting antigen (A). The spleen cells from immunized mice were also collected and IFN-γ-secreting cells were analyzed (B).

### DETAILED DESCRIPTIONOF THE PREFERRED EMBODIMENTS

The detailed description provided below in connection with the appended drawings is intended as a description of the present disclosure and is not intended to represent the only forms in which the present disclosure may be constructed or utilized.

### 1. Definitions

Unless otherwise indicated, the term “substituted,” when used to describe a chemical structure or moiety, refers to a derivative of that structure or moiety wherein one or more of its hydrogen atoms is substituted with an atom, chemical moiety or functional group such as, but not limited to, —OH, alkyl (e.g., methyl, ethyl, propyl, t-butyl and etc), alkoxy, halo (e.g., fluoro, chloro, bromo, and iodo), haloalkyl, cycloalkyl, phenyl, biphenyl and —O-phenyl.

An atom, moiety, or group described herein may be unsubstituted or substituted, as valency permits, unless otherwise provided expressly. The term “optionally substituted” refers to substituted or unsubstituted.

The term “salt” refers herein as a salt which is formed by the interaction of an acid (e.g., the present compound) with a base, including organic or inorganic types of bases. Salts of inorganic base may include, for example, alkaline metals (e.g., sodium hydroxide, potassium hydroxide and etc), alkaline earth metals (e.g., calcium, magnesium, and aluminum), and ammonia. Salts of organic base may be alkylamine and etc.

The term “solvate” refers to forms of the compound that are associated with a solvent, usually by a solvolysis reaction. This physical association may include hydrogen bonding. Conventional solvents include water, methanol, ethanol, acetic acid, dimethyl sulfoxide (DMSO), tetrahydrofuran (THF), diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates.

It should also be noted that if the stereochemistry of a structure or a portion of a structure is not indicated with, for example, bold or dashed lines, the structure or the portion of the structure is to be interpreted as encompassing all stereoisomers of it. Similarly, names of compounds having one or more chiral centers that do not specify the stereochemistry of those centers encompass pure stereoisomers and mixtures thereof. Moreover, any atom shown in a drawing with unsatisfied valences is assumed to be attached to enough hydrogen atoms to satisfy the valences.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R-and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Unless otherwise indicated, “an effective amount” of a compound is an amount sufficient to provide a therapeutic or prophylactic benefit in the treatment or management of a disease or condition, or to delay or minimize one or more symptoms associated with the disease or condition; or to prevent a disease or condition, or one or more symptoms associated with the disease or condition, or prevent its recurrence. An effective amount of a compound is an amount of a therapeutic agent, alone or in combination with other therapies, which provides a therapeutic benefit in the treatment or management of the disease or condition, or a prophylactic benefit in the prevention of the disease. The term “an effective amount” can encompass an amount that improves overall therapy, reduces or avoids symptoms or causes of a disease or condition, or enhances the therapeutic efficacy of another therapeutic agent; or improves overall prophylaxis or enhances the prophylactic efficacy of another prophylactic agent.

Unless otherwise indicated, the terms “treat,” “treating” and “treatment” contemplate an action that occurs while a patient is suffering from the specified disease or disorder, which reduces the severity of the disease or disorder, or one or more of its symptoms, or retards or slows the progression of the disease or disorder.

The term “adjuvant” refers to an agent (e.g., the compound of the present disclosure) capable of stimulating an immune response to an administered immunogen (e.g., a virus). The increased immune response may be an increase in the immunogen specific IgG levels in the blood post administration as compared to the same immunogen administered without any adjuvant. Alternatively, the adjuvant might not increase immunogen specific IgG levels, but results in an increase in T cells or triggers an innate immune response.

The term “subject” or “patient” is used interchangeably herein and is intended to mean a mammal including the human species that is susceptible to infection by a virus. The term “mammal” refers to all members of the class Mammalia, including humans, primates, domestic and farm animals, such as rabbit, pig, sheep, and cattle; as well as zoo, sports or pet animals; and rodents, such as mouse and rat. Further, the term “subject” or “patient” intended to refer to both the male and female gender unless one gender is specifically indicated. Accordingly, the term “subject” or “patient” comprises any mammal which may benefit from the treatment method of the present disclosure. Examples of a “subject” or “patient” include, but are not limited to, a human, rat, mouse, guinea pig, monkey, pig, goat, cow, horse, dog, cat, bird, and fowl. In a preferred embodiment, the subject is a human.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The singular forms “a”, “and”, and “the” are used herein to include plural referents unless the context clearly dictates otherwise.

### 2. Novel NOD1 Agonists

The present disclosure is directed to novel NOD1 agonists having adjuvant properties, vaccines comprising the NOD1 agonists, and the use of the NOD1 agonists in prophylaxis or therapy for influenza.

Thus, in one aspect, the present disclosure encompasses compounds of formula (I), a pharmaceutically acceptable salt, solvate or stereoisomer thereof,

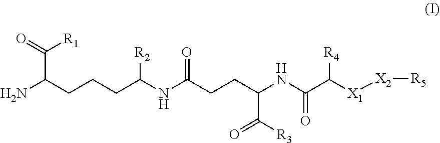

wherein,
X₁ and X₂ are independently nil, —NH or —(C═O);
R₁ and R₃ are independently hydroxyl or alkoxy;
R₂ is —COOH or —CH₂OH;
R₄ is hydrogen or C₁₋₆ alkyl;
R₅ is hydrogen or C₁₋₂₀ alkyl optionally substituted with a substituent selected from the group consisting of cycloalkyl, phenyl, biphenyl and —O-phenyl; and
the phenyl, the biphenyl or the —O-phenyl is optionally substituted with one or more halo, alkyl, or alkoxy.

According to some embodiments of the present disclosure, in the formula (I), X₁ is nil, X₂ is —NH, R₁ is hydroxy, R₂ is methoxy, R₃ is hydroxy, R₄ is isopropyl or isobutyl, and R₅ is hydrogen. Exemplary compounds in these embodiments include, but are not limited to,

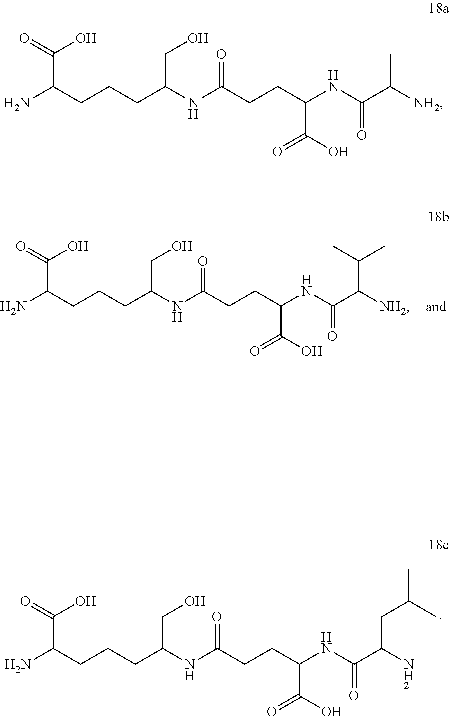 [SMILES: CC(N)C(=O)NC(CCC(=O)NC(CO)CCCC(N)C(=O)O)C(=O)O ; CC(C)C(N)C(=O)NC(CCC(=O)NC(CO)CCCC(N)C(=O)O)C(=O)O ; CC(C)CC(N)C(=O)NC(CCC(=O)NC(CO)CCCC(N)C(=O)O)C(=O)O]

According to some embodiments of the present disclosure, particular compounds are of formula (II),

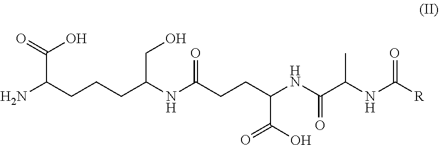

wherein,
R is C₁₋₂₀ alkyl optionally substituted with a substituent selected from the group consisting of cycloalkyl, phenyl, biphenyl and —O-phenyl; and
the phenyl, the biphenyl or the —O-phenyl is optionally substituted with one or more halo, alkyl, or alkoxyl.

Exemplary compounds of formula (II) include, but not limited to, the followings,

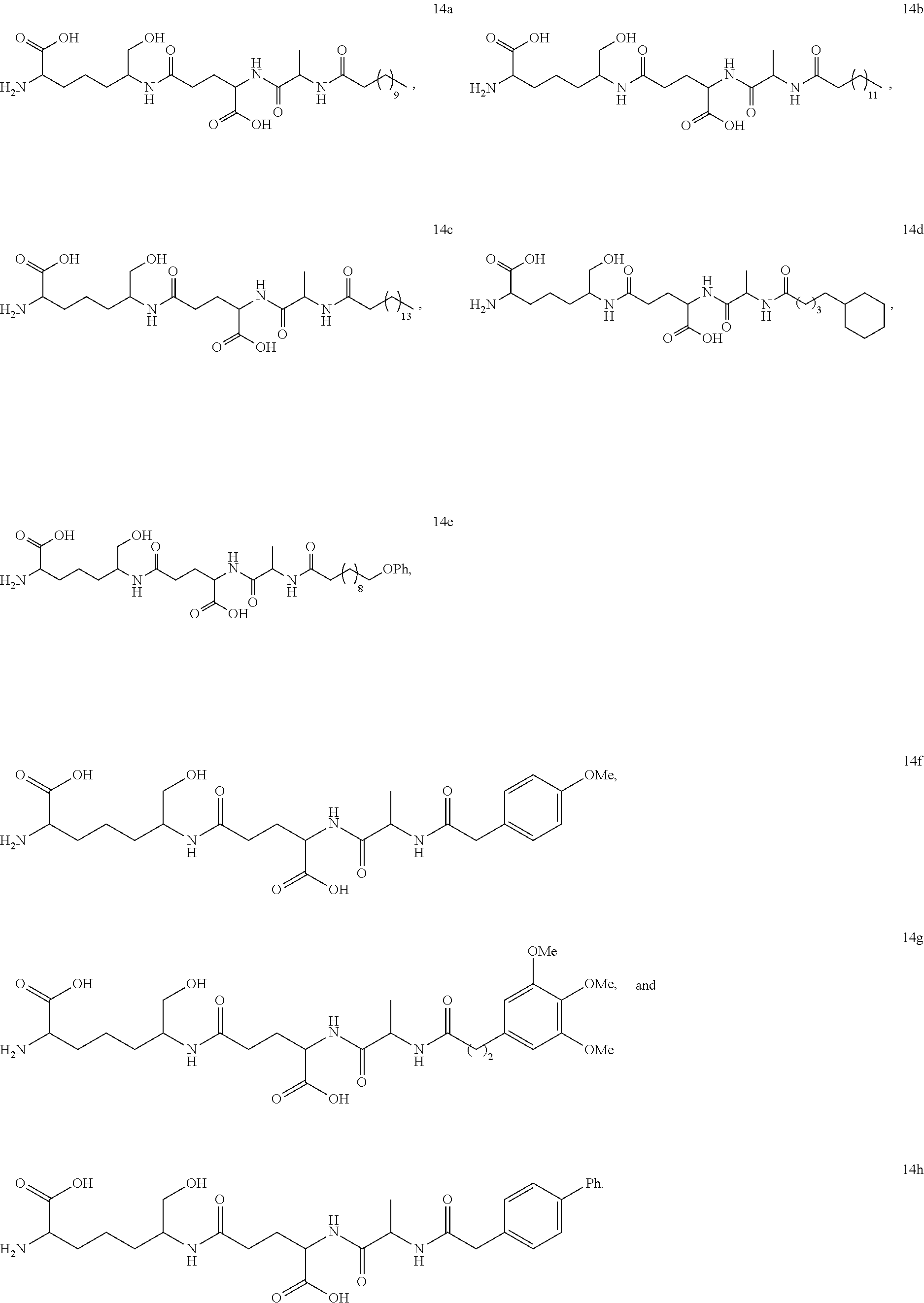

According to one preferred embodiment of the present disclosure, the compound of formula (II) is

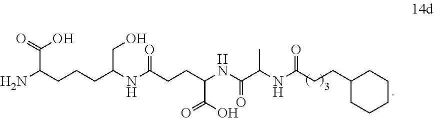

According to another preferred embodiment of the present disclosure, the compound of formula (II) is

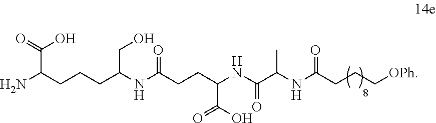

According to further embodiments of the present disclosure, particular compounds are of formula (III),

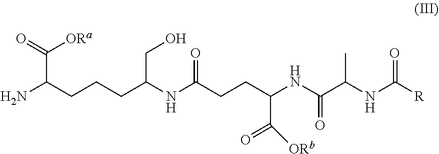

wherein,
R^{a} and R^{b} are independently C₁₋₆alkyl;
R is C₁₋₂₀ alkyl optionally substituted with a substituent selected from the group consisting of cycloalkyl, phenyl, biphenyl and —O-phenyl; and
the phenyl, the biphenyl or the —O-phenyl is optionally substituted with one or more halo, alkyl, or alkoxyl

Exemplary compounds of formula (III) include, but not limited to, the followings,

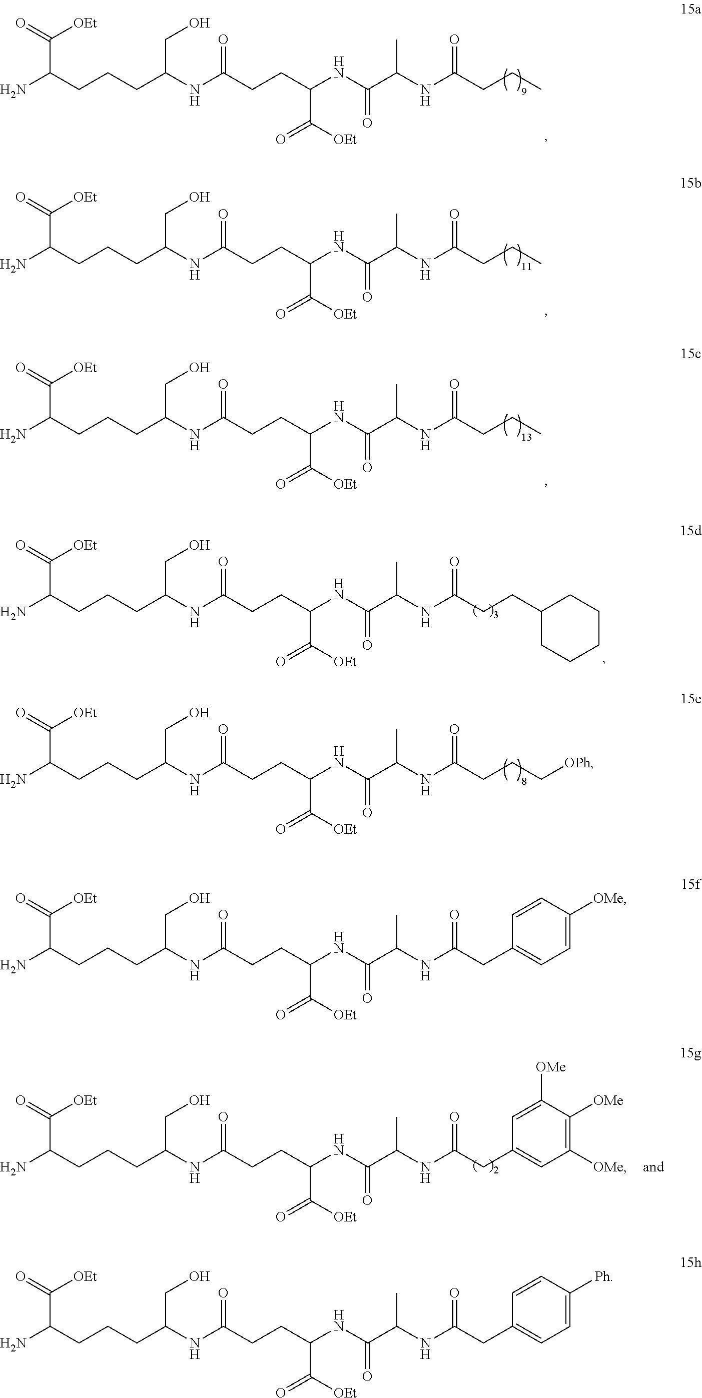

According to further embodiments of the present disclosure, particular compounds are of formula (IV),

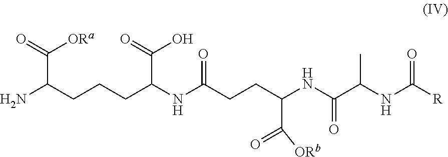

wherein,
R^{a} and R^{b} are independently C₁₋₆ alkyl;
R is C₁₋₂₀ alkyl optionally substituted with a substituent selected from the group consisting of cycloalkyl, phenyl, biphenyl and —O-phenyl; and
the phenyl, the biphenyl or the —O-phenyl is optionally substituted with one or more halo, alkyl, or alkoxyl.

Exemplary compounds of formula (IV) include, but not limited to, the followings,

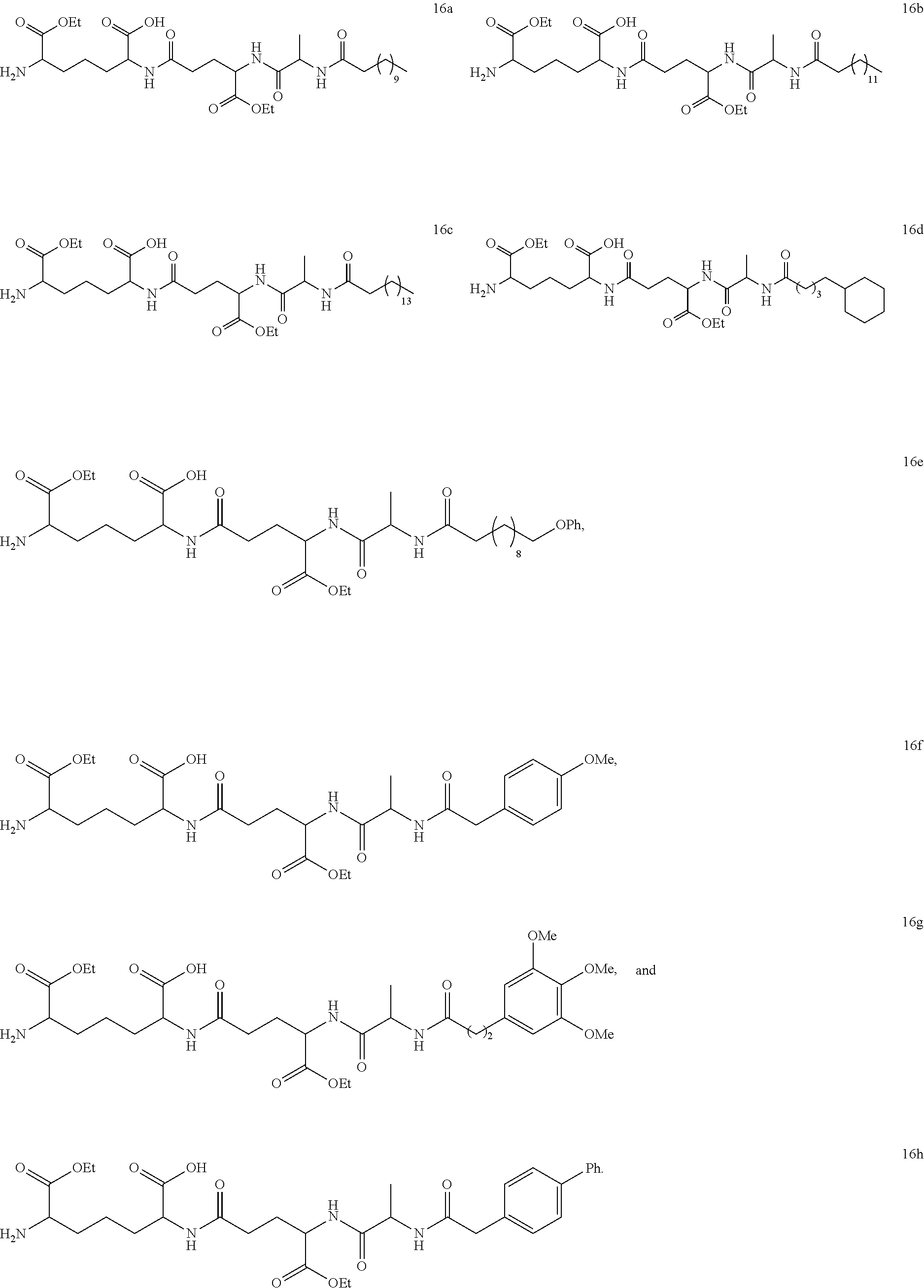

According to still further embodiments of the present disclosure, particular compounds are of formula (V),

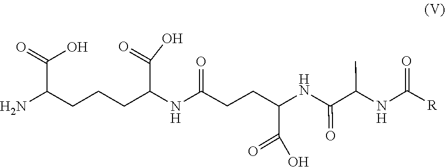

wherein,
R is C₁₋₂₀ alkyl optionally substituted with a substituent selected from the group consisting of cycloalkyl, phenyl, biphenyl and —O-phenyl; and
the phenyl, the biphenyl or the —O-phenyl is optionally substituted with one or more halo, alkyl, or alkoxyl.

Exemplary compounds of formula (V) include, but not limited to, the followings,

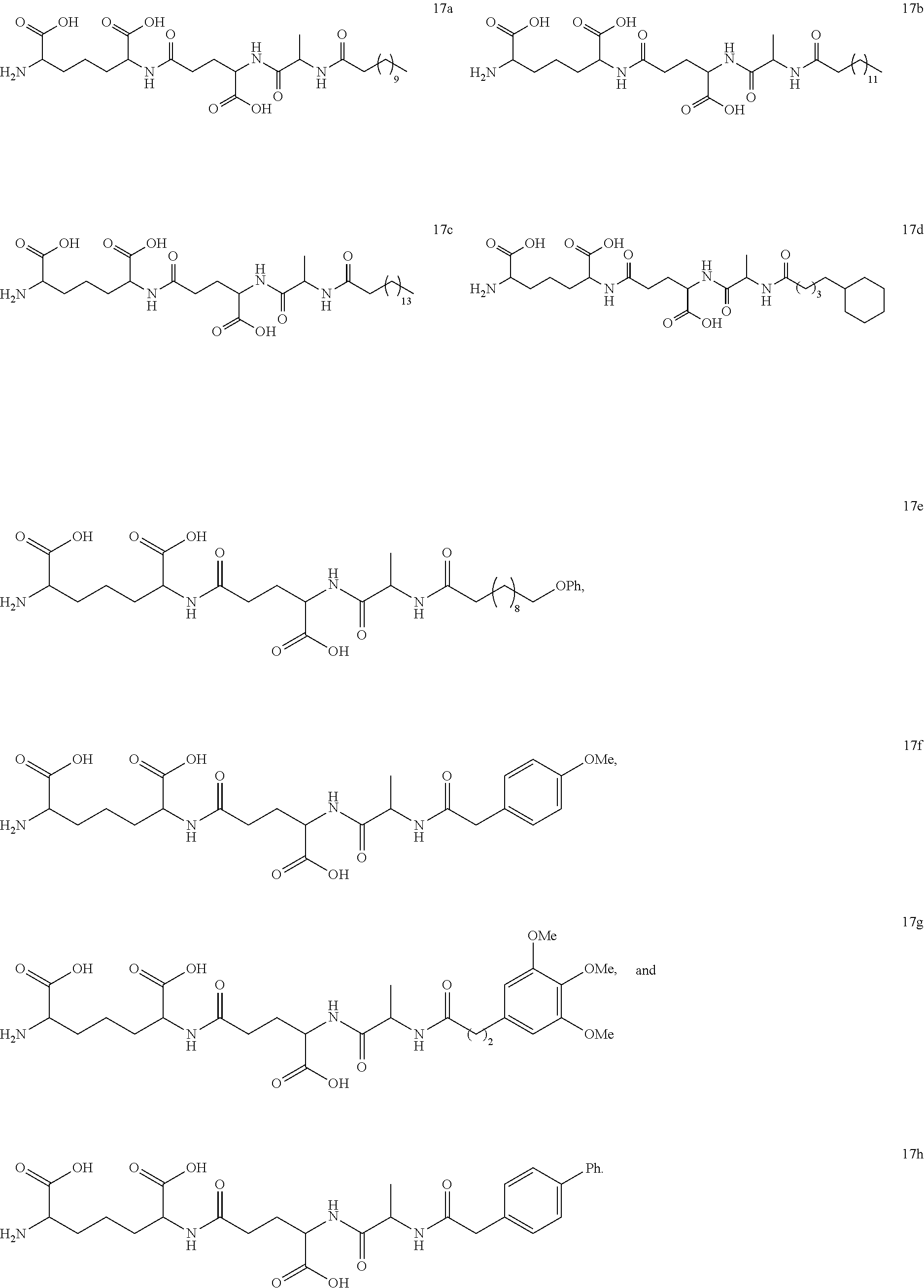

According to other embodiments of the present disclosure, particular compounds are of formula (VI),

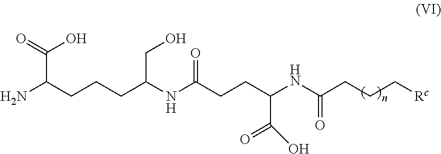

wherein,
n is an integral between 3 to 10;
R^{c} is H, cycloalkyl, phenyl, biphenyl or —O-phenyl; and
the phenyl, the biphenyl or the —O-phenyl is optionally substituted with one or more halo, alkyl, or alkoxyl.

Exemplary compounds of formula (VI) include, but not limited to, the followings,

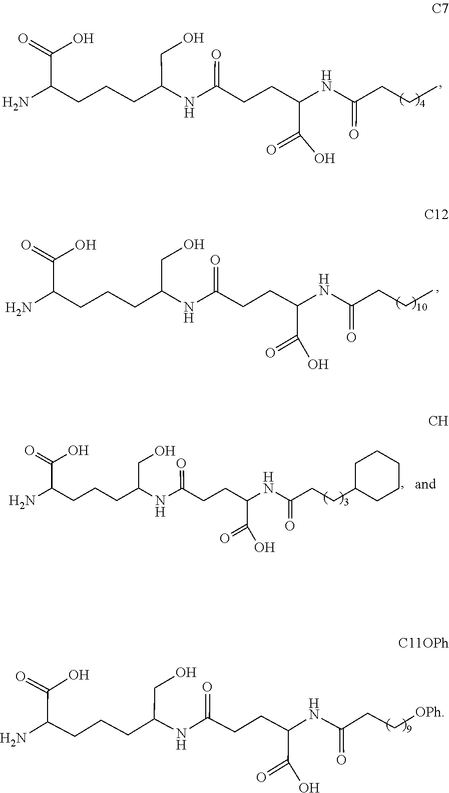

Compounds of the invention contain one or more stereocenters, thus can exist as racemic mixtures of enantiomers or mixtures of diastereomers. This invention thus encompasses stereomerically pure forms of such compounds, as well as mixtures of those forms. Stereoisomers may be asymmetrically synthesized or resolved using standard techniques such as crystallization, chromatography, and the use of a resolving agent. One preferred way of separating enantiomers from a racemic mixture is by use of preparative high performance liquid chromatography (HPLC) by chiral column. Alternatively, the racemic may be separated into its enantiomers by reacting with an optically active form of a resolving agent in the presence of a solvent. Depending on the optical form of the resolving agent, one of the two enantiomers is separated out as an insoluble salt with high yield and high optical purity, while the opposite enantiomer remains in the solution.

The present invention thus further encompasses stereoisomeric mixtures of compounds disclosed herein. It also encompasses configurational isomers of compounds disclosed herein (e.g., cis- and trans-isomers, whether or not involving double bonds), either in admixture or in pure or substantially pure form.

### 3. Vaccines

The compounds of formula (I) are found to exhibit adjuvant activity against an immunogen. Thus, in another aspect, the present disclosure encompasses a vaccine that comprises any one of the compounds of formula (I), a pharmaceutically acceptable salt, solvate or stereoisomer thereof; and an immunogen; in which the immunogen is capable of eliciting an immune response against a respiratory virus, and the compound of formula (I), the pharmaceutically acceptable salt, solvate or stereoisomer thereof is capable of enhancing the immune response elicited by the immunogen.

According to embodiments of the present disclosure, the immunogen is an antigen that elicits an immune response against an influenza virus, such as an avian influenza virus (e.g., H5N1, H7N9 and etc.), a seasonal influenza virus and the like.

The antigen may be a live attenuated virus, a non-live flue antigen, or a combination thereof. A non-live flue antigen suitable for use in the present disclosure may be selected from the group consisting of split virus antigens (e.g., split flu antigens), subunit antigens prepared from whole virus or recombinantly expressed, or inactivated whole virus which may be chemically inactivated by any suitable means including, for example, by treating with formaldehyde, formalin, β-propiolactone or otherwise inactivated by ultraviolet light or heat inactivation. According to embodiments of the present disclosure, the immunogen is an influenza antigen derived from split flu virus process. Traditionally, split flu virus is produced by treating the virus with a solvent/detergent, such as tri-n-butyl phosphate, diethyl ether in combination with Tween™. Other splitting agent include detergents, proteolytic enzymes, and bile salts. Detergents that can be used as splitting antigens include cationic detergents (e.g., cetyl trimethyl ammonium bromide (CTAB)), non-ionic detergents (e.g., Triton X-100, Triton N-101, and etc.), and others (e.g., laurylsulfate, and taurodeoxycholate).

Alternatively, antigens suitable for use in the present disclosure may also include, for example, proteins (e.g., neuraminidase (NA)), recombinant proteins, peptides, polysaccharides, glycoproteins (e.g., hemagglutinin (HA)), and lipopolysaccharides. According to preferred embodiments of the present disclosure, the antigen is a glycoprotein (e.g., HA) found on the surface of an influenza virus. The immunogen may be provided in purified or an unpurified form. Preferably, the immunogen is provided in a purified form.

Preferably, the immunogen is present in an amount sufficient to induce an immune response without significant adverse side effects. Such amount will vary depending on which immunogen is used and the type and amount of the present compound of formula (I) included in the vaccine. Typically, a vaccine will comprise immunogen in an amount of about 1 to 1,000 μg/mL, more preferably about 3 to 300 μg/mL, and most preferably about 10 to 100 μg/mL, as measured by single radial immunodiffusion (SRID) assay.

Advantageously, the present compound of formula (I) will enhance the immunogenicity of a vaccine comprising the immunogen described above. In one preferred embodiment, in addition to the immunogen described above, the vaccine also includes

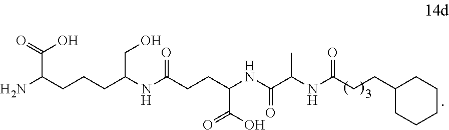

In another preferred embodiment, in addition to the immunogen described above, the vaccine also includes

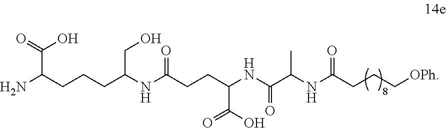

Optionally, the vaccine may further include another adjuvant in addition to the present compound of formula (I). Examples of such adjuvant suitable for inclusion in the present vaccine include those well known in the art, such as complete Freund's adjuvant (CFA), incomplete Freund's adjuvant (IFA), squalene, squalene, alum, and various types of oils, all of which are well known in the art, and are available commercially from various sources. According to some embodiments of the present disclosure, in addition to the immunogen and the compound of formula (I), the vaccine also includes alum as the additional adjuvant.

Depending on the route of administration, the vaccine may take the form of a solution, a suspension, an emulsion, a powder, or the like. A vaccine of the present disclosure may be administered intranasally or through parenteral administration, such as through sub-cutaneous injection, intra-muscular injection, intravenous injection, intraperitoneal injection, or intradermal injection to a mammal, such as humans, primates and etc. As the present compound of formula (I) has adjuvant property in vaccines for respiratory viruses, the vaccines thus can be delivered locally to the respiratory system, for example, to the nose, sinus cavities, sinus membranes or lungs. The vaccines containing the present compound of formula (I) can be delivered to the respiratory system in any suitable manner, such as by inhalation via the mouth or intranasally. The vaccines can be dispersed as powdered or liquid nasal spray, suspension, nose drops, a gel or ointment, through a catheter, by syringe, or by submucosal infusion. The vaccines may be delivered in the form of an aerosol spray using a pressurized pack or a nebulizer and a suitable propellent (e.g., dichlordifluoromethane, trichlorofluoromethane, dichlorotetrafluoroethane, or carbon dioxide). In the case of a pressurized aerosol, the dosage unit may be controlled by providing a valve to deliver a metered amount. Capsules and cartridges of, for example, gelatin for use in an inhaler or insufflator may be formulated to contain a powder mix of the vaccine and a suitable powder base (e.g., starch or lactose). Examples of a propellent suitable for use in an aerosol formulation include, but are not limited to, compressed air, nitrogen, carbon dioxide, and a hydrocarbon base low boiling point solvent. Preferably, the active ingredients (i.e., the immunogen and the compound of the present disclosure) are micronized so as to permit inhalation of substantially all of the active ingredients into the lungs upon administration of the dry powder formulation.

### 4. Method of Use

The present invention also encompasses a method for immunizing a subject against a viral respiratory infection. The method comprises the step of administering to the subject an effective amount of the vaccine described above, which comprises an immunogen capable of eliciting an immune response against a viral respiratory infection, and the compound of formula (I) capable of enhancing the immune response elicited by the immunogen.

The vaccines are preferably administered prophylactically. For instance, administration of the vaccine may be commenced before or at the time of infection or at the time the subject is exposed to a respiratory virus, and optionally continued until the virus is no longer present or active in the respiratory tract. In some embodiments, the vaccines are administered at least one week prior to exposure to the respiratory virus (e.g., influenza virus). In other embodiments, the vaccines are administered at least two weeks, or at least one month prior to exposure to the respiratory virus.

Advantageously, the vaccines are administered as a single dose or as divided doses at appropriated intervals, for example, as 2, 3, 4 or more doses per day. Alternatively, the vaccines may be administered on day one, then followed by one or more booster doses spaced as desired thereafter, although the booster doses are not required for the vaccine to protect the subject against infection. Typically, an initial vaccination is administered, followed by a booster of the same vaccine approximately 7 to 14 days later.

The present invention will now be described more specifically with reference to the following embodiments, which are provided for the purpose of demonstration rather than limitation. While they are typically of those that might be used, other procedures, methodologies, or techniques known to those skilled in the art may alternatively be used.

### EXAMPLES

### Materials and Methods

### Cell Culture

Each type of cells used in the present study were grown in manufactures' suggested medium supplemented with 10% heat-inactivated fetal bovine serum, 100 units/mL penicillin, and 100 μg/mL streptomycin, at 37° C. in a humidified 5% CO₂ incubator.

Measurement of NOD1-induced NF-κB Activation Assay

HEK-Blue™ hNOD1 Cells (Invivogen; San Diego, CA, U.S.) were cultured in accordance with the manufacturer's instructions. HEK-Blue™ hNOD1 Cells was assayed for NF-κB transcriptional activity changes upon incubation (3.6×10⁵ cells/mL) with iE-DAP, C12-iE-DAP, Tri-DAP (Invivogen, San Diego, CA, U.S.) and other NOD1 agonistic compounds (0.1-10 μM) for 16 h. Secreted embryonic alkaline phosphatase (SEAP) activity was determined in the supernatant in accordance with the manufacturer's instructions. An amount of 20 μL of SEAP-inducer compound or negative control was added to 180 μL of cells in HEK-Blue™ detection medium and incubated at 37° C. for 16 h. Absorbance was measured on a SpectraMax M5 (Molecular Devices, Sunnyvale, CA, U.S.) at 640 nm.

### Determination of HA-specific Antibodies by ELISA

HA-specific antibody titers were detected by ELISA using HA WSN (Sino biological, Beijing, P.R. China) as the substrates. Ninety-six-well ELISA plate (Greiner bio-one, Frickenhausen, Germany) was coated with 100 μl of protein diluted in ELISA coating buffer, 100 mM sodium bicarbonate (pH 8.8), at a concentration of 5 μg/ml per well and covered with a plastic sealer at 4° C. for overnight. After the plates were blocked with 1% BSA in TBST (137 mM NaCl, 20 mM Tris-base, 0.05% Tween 20, pH 7.4) at 37° C. for 1 h and washed 3 times with TBST, the plates were incubated with 200 μl of mouse serum in 2-fold serial dilutions at 37° C. for 2 h. After serum was moved and the plate was washed 6 times, HA-specific IgG was monitored by using 200 μl of secondary HRP-labeled anti-mouse antibody (1:8000) (PerkinElmer, Waltham, MA, U.S.). After 1 h of incubation at 37° C., the plates were washed 6 times with TBST and developed with 100 μl of the Super Aquablue ELISA substrate (eBioscience, San Diego, CA, U.S.) for 1 min. The reaction was stopped with the addition of 100 μL of 0.625 M oxalic acid. The absorbance of wells was measured at 405 nm using a SpectraMax M5 (Molecular Devices, Sunnyvale, CA, U.S.). The endpoint antibody titer was defined as the highest dilution of serum to produce an absorbance 2.5 times higher than the optical absorbance (OD) produced by the negative control (preimmune serum). The background endpoint antibody titer was assigned as less than 1:50.

### Enzyme-linked Immunospot (ELISpot) Assay

ELISPOT plates were coated with anti-mouse IFN-γ (Mabtech AB, Stockholm, Sweden). The plates were washed four times and incubated for 30 min with RPMI-1640 supplemented with 10% Fetal bovine serum (Gibco). For the detection of IFN-γ secreting cells from immunized mice, splenocytes were collected and cultured at 5×10⁵ per well at 37° C. in 5% CO₂ for 24 h with specific peptides from HA for restimulation. The cells were removed and incubated with biotinylated anti-mouse IFN-γ specific antibody. The plates were washed five times before the addition of streptavidin-ALP conjugate and developed with ready-to-use BCIP/NPT substrate. Following drying, the number of resulting spots was analyzed by use of an Immune Spot Reader (Cellular Technology Ltd.).

### Example 1 Chemical Synthesis of Compounds of Formula (I)

The present compounds of formula (I) were synthesized in accordance with procedures described in Schemes 1 to 2.

### 1.1 N-Substituted Tripeptides 13a-h and 14a-h

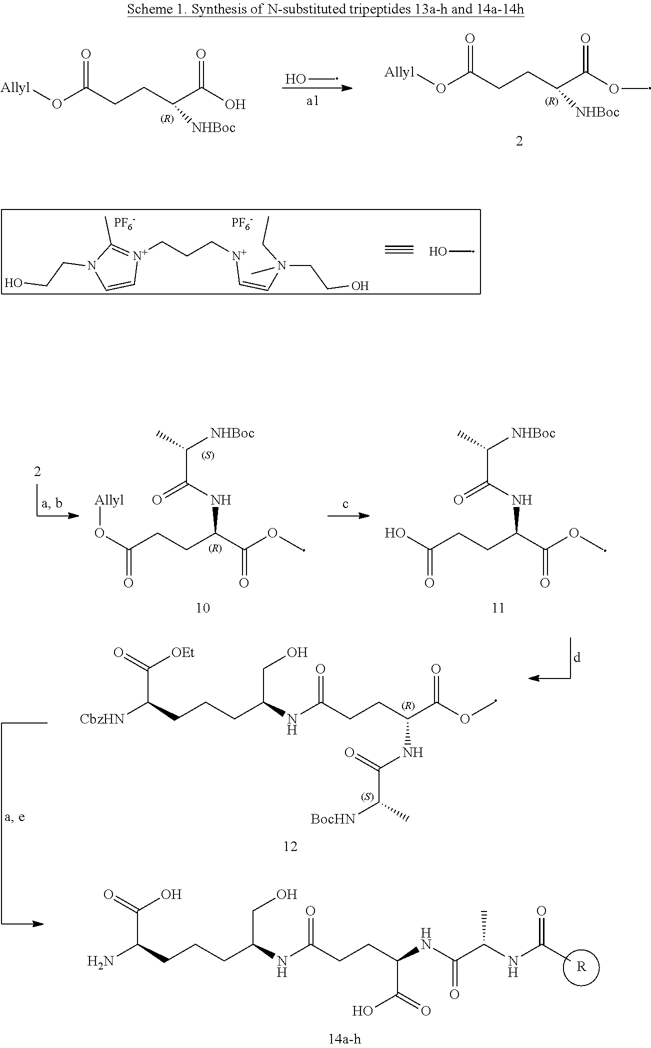

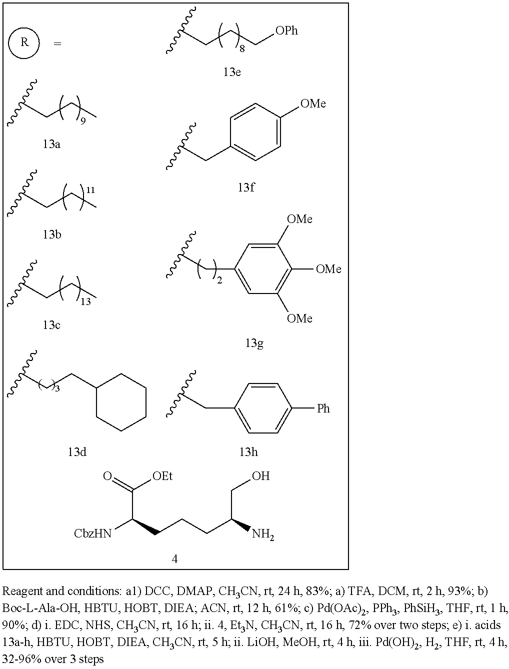

Hydroxy-advanced ionic liquid matrix (AILM-OH) was conjugated to the protected L-glutamic acid 1 under N,N′-dicyclohexylcarbodiimide (DCC)/4-Dimethylaminopyridine (DMAP) coupling conditions to generate AILM-bound 2 in 80% yield. The N-Boc group of 2 was then removed, and the resulting amine coupled with Boc-L-alanine under mild conditions (Hexafluorophosphate benzotriazole tetramethyl uronium (HBTU)/Hydroxybenzotriazole (HOBt)/Diisopropylethylamine (DIEA)) gave 10 (61%), which underwent allyl deprotection using Pd (OAc)₂/Ph₃P/PhSiH₃ to give 11 in good yield (90%). The dipeptide intermediate 11 was converted to the corresponding NHS-ester, and then immediately coupled with 4 to generate the N-Boc protected tripeptide 12, a common intermediate for the divergent synthesis of our target library. A sequence of steps including (i) N-Boc deprotection, (ii) conjugation with various lipophilic acids (13a-h) via an amide bond formation, (iii) base-catalyzed ester hydrolysis with concomitant cleavage of the Cbz protected tripeptides from AILM, and (iv) catalytic hydrogenation (Pd (OH)₂/H₂) to remove the Cbz protecting group, were performed to easily produce desired tripeptides 14a-h from 12. Notably, any side products could be removed by simple washing or extraction, and no tedious column chromatography was needed during ionic liquid-supported synthesis (ILSS) (Scheme 1).

Compound 12. ¹H NMR (600 MHZ, CD3OD) δ 7.52-7.65 (m, 4H), 7.26-7.47 (m, 10H), 5.09 (m, 4H), 3.59-4.64 (m, 24H), 3.55-3.40 (m, 4H), 2.52-2.83 (m, 8H), 1.05-2.52 (m, 50H). ¹³C NMR (150 MHz, CD3OD) δ 176.8, 176.3, 174.3 (×2), 174.1 (×2), 173.9, 172.5, 158.7, 158.6, 158.0 (×2), 146.6 (×2), 138.2, 138.1, 129.5 (× 4), 129.0(×2), 128.8 (×2), 128.7 (×2), 123.5, 123.1, 122.5, 122.2, 80.9 (×2), 67.6, 67.5, 65.1 (x×2), 64.0 (×2), 62.4, 62.3, 55.5 (×2), 55.1, 54.2, 52.5, 52.4 (×2), 52.3, 51.8 (×2), 46.3 (×2), 32.9 (×2), 32.4, 32.3, 31.4 (×2), 30.2, 28.7 (× 6), 27.8 (×2), 23.4 (×2), 18.1 (×2), 14.5, 14.4, 9.9 (×2); HRMS (ESI) m/z calculated for C₇₅H₁₁₄N₁₂O₂₂²⁺: 767.4080 [M]²⁺; found: 767.4083.

### 1.2 N-substituted tripeptides 15a-h, 16a-h and 17a-h

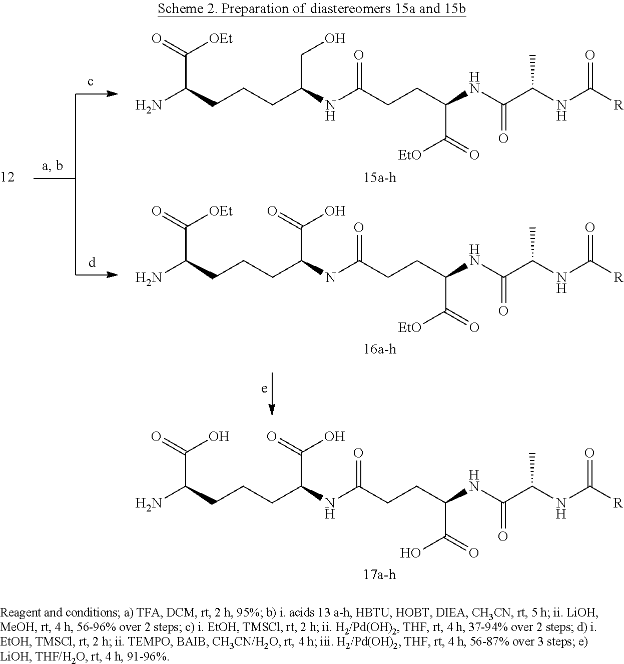

The two carboxylic acid groups in AILM-bound intermediate 12 were converted to their corresponding ethyl esters, and 2,6-diamnio-7 hydroxyheptanoic acid (DAHH) was converted to 2,6-diaminopimelic acid (DAP) (Scheme 2). The desired N-substituted tripeptides were cleaved from matrix, followed by esterification through treatment with trimethylsilyl chloride and ethanol, and then hydrogenolysis for N-Cbz deprotection to give the series of tripeptides 15a-h as shown in scheme 2. Next, with a slight modification of transformation sequences, and introduction of TEMPO/BAIB oxidation, the series 16a-h bearing two ethyl esters was obtained. Finally, base-catalyzed hydrolysis of 16a-h gave the corresponding 17a-h (Scheme 2).

### 1.3 Compounds C7, C12, CH and C110Ph

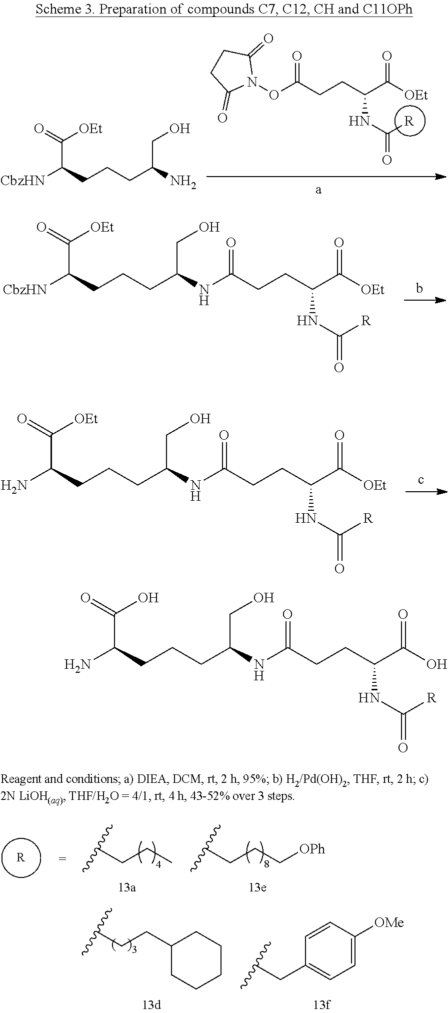

### Compound C7

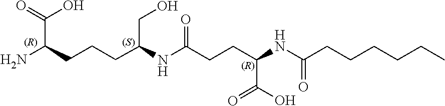

White solid (49 mg, 43% after 3 steps); [α]_{D}²²=−11.8 (c =0.95, CH₃OH), TLC (n-PrOH/H₂O =3/1) R_{f}=0.5; 1H NMR (600 MHZ, MeOD) δ 4.21-4.28 (m, 1H), 3.86-3.93 (m, 1H), 3.48-3.60 (m, 3H), 2.12-2.05 (m, 4H), 1.93-2.01 (m, 2H), 1.72-1.82 (m, 1 H), 1.54-1.67 (m, 4H), 1.40-1.49 (m, 2H), 1.29-1.37 (m, 6H), 0.90 (t, J=6.3 Hz, 3H); ¹³C NMR (150 MHz, MeOD) δ 178.1, 175.6, 175.5, 174.5, 65.1, 56.1, 55.4, 52.3, 37.2, 33.6, 32.7, 32.1, 31.7, 30.09, 30.02, 26.8, 23.5, 22.8, 14.4 ppm; HRMS (ESI-OTF) calculated for C₂₇H₅₀N₄O₈ [M−H]⁻: 557.3556, found 557.3573.

### Compound C12

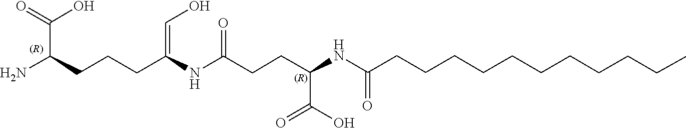

White solid (54 mg, 75% after 3 steps); [α]_{D}²²=−11.4 (c=0.47, CH₃OH), TLC (n-PrOH/H_{20=3/1}) R_{f}=0.5; 1H NMR (600 MHZ, MeOD) 8 4.23-4.27 (m, 1H), 3.85-3.94 (m, 1H), 3.47-3.59 (m, 3H), 2.21-2.32 (m, 4H), 2.06-2.14 (m,1H), 1.93-2.01 (m, 2H), 1.72-1.82 (m, 1H), 1.41-1.50 (m, 4H), 1.41-1.49 (m, 2H), 1.27-1.36 (m, 16H), 0.90 (t, J=6.9 Hz, 3H); ¹³C NMR (150 MHZ, MeOD) 8 178.0, 175.6 (×2), 174.5, 65.1, 56.1, 55.3, 52.3, 37.2, 33.6, 33.0, 32.1, 31.7, 30.78, 30.75, 30.6, 30.5, 30.48, 30.44, 29.9, 26.9, 23.7, 22.8, 14.4 ppm; HRMS (ESI-TOF) calculated for C₂₉H₅₄N₄O₈ [M−H]⁻585.3869, found 585.3865.

### Compound CH

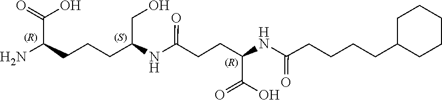

White solid (47 mg, 52% after 3 steps); [α]_{D}²²=−9.70 (c=0.62, CH₃OH); TLC (n-PrOH/H₂O=3/1) R_{f}=0.5; 1H NMR (600 MHZ, MeOD) δ 4.21-4.28 (m, 1H), 3.85-3.93 (m, 1H), 3.46-3.56 (m, 3H), 2.19-2.32 (m, 4H), 2.07-2.15 (m, 1H), 1.92-2.03 (m, 2H), 1.52-1.83 (m, 9H), 1.49-1.40 (m, 2H), 1.12-1.38 (m, 9H), 0.82-0.94 (m, 2H); ¹³C NMR (150 MHz, MeOD) δ 178.0, 175.6, 175.5, 174.5, 65.1, 56.1, 55.2, 52.3, 38.8, 38.4, 37.2, 34.5 (×2), 33.6, 32.1, 31.7, 29.9, 27.8, 27.6, 27.5 (×2), 27.2, 22.7 ppm. HRMS (ESI-TOF) calculated for C₃₁H₅₇N₄O₈ [M−H]⁻613.4182, found 613.4148.

### Compound C110Ph

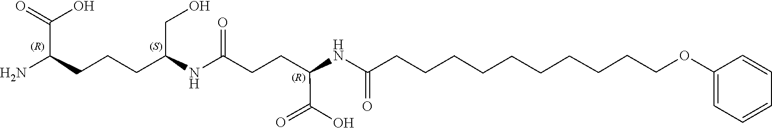

White solid (40 mg, 47% after 3 steps); [α]_{D}²²=−11.3 (c=0.53, CH₃OH); TLC (n-PrOH/H_{20=3/1}) R_{f}=0.5; 1H NMR (600 MHZ, CD3OD) 8 7.20-7.29 (m, 2H), 6.83-6.93 (m, 3H), 4.21-4.30 (m, 1H), 3.94 (t, J=6.7 Hz, 2H), 3.85-3.90 (m, 1H), 2.21-2.29 (m, 4H), 2.07-2.24 (m, 1H), 1.93-2.01 (m, 2H), 1.72-1.81 (m, 3H), 1.53-1.66 (m, 4H), 1.41-1.5 (m, 4H), 1.31-1.38 (m, 9 H); ¹³C NMR (150 MHz, CD3OD) δ 177.8, 15.6, 175.5, 174.5, 160.5, 130.3 (×2), 121.4, 115.4 (×2), 68.8, 65.1, 56.0, 55.1, 52.3, 37.2, 33.5, 32.1, 31.7, 30.6, 30.5, 30.47, 30.43, 29.8, 27.1, 26.9, 22.7 ppm; HRMS (TOF-ESI) calculated for C26H₄₆N408 ([M+H]⁺): 543.3394. Found 543.3389.

### 1.4 General Procedure for the Preparation of Compounds 6-9 (Series 18)

To a solution of 5 (150 mg, 0.09 mmol) in DCM (1 mL) was added the mixture of TFA (500 μL) in DCM (500 μL) dropwise at rt for 30 min. The solvent was removed, and the residue was washed with ether (10 mL×3) and concentrated to give amine intermediate as a yellow solid. To a solution of the above amine in ACN (2 mL) were added Cbz-L-alanine (60 mg, 0.27 mmol), HBTU (102 mg, 0.27 mmol) and HOBT (37 mg, 0.27 mol), and DIEA (70 mg, 540 mmol) was added. After the reaction was stirred at rt for 12 h, the solvent was removed, the crude was dissolved in DCM (10 mL), washed with 1 N HCl_{(aq)} (5 mL×3), and concentrated. The resulting residue was dissolved in DCM (2 mL) and added ether until the product was completely precipitated out. The precipitated product and phase separation were accomplished by centrifugation and decantation. The precipitate was further washed with ether (5 mL×2), then dried under vacuum to give an intermediate. To a solution of the resulting intermediate in MeOH (1 mL) was added 2 N LiOH_{(aq)} (450 μL, 0.9 mmol) dropwise at 0° C. After the reaction was stirred for 4 h, the reaction mixture was neutralized by Dowex® resin (H⁺ form) to pH<7. Then the solvent was filtered, concentrated, and purified by cc (CHCl₃/MeOH/H₂O=60/25/4) to get Cbz-intermediate as a white solid. To a solution of Cbz-intermediate (90 mg, 0.14 mmol) in THF (1 mL) was added palladium hydroxide on activated charcoal (4.2 mg, 0.03 mmol). The reaction mixture was vigorously stirred under an atmosphere of hydrogen for 4 h. The mixture was filtered through a pad of celite, concentrated, and purified by cc (n-PrOH/H₂O=5/1) to give compounds 6-9 (40-62% over 4 steps) as white solids.

### Compound 6

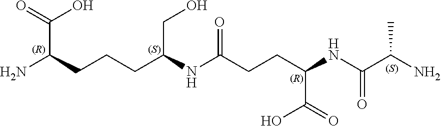

A white solid (37 mg, 54% over 4 steps from 5); [α]_{D}²²=23.7 (c=0.13, CH₃OH/H₂O=4/1). ¹H NMR (600 MHZ, D20) δ 4.15-4.28 (1H, m), 4.08-4.12 (1H, m), 3.85-3.91 (1H, m), 3.70-3.74 (1H, m), 3.55-3.60 (1H, m), 3.47-3.51 (1H, m), 2.27-2.37 (2H,m), 2.08-2.16 (1H, m), 1.77-2.99 (3H, m), 1.67-1.63 (1H, m), 1.53 (3H, d, J=7.09 Hz), 1.47-1.36 (3H, m); ¹³C NMR (150 MHZ, D20): d=177.8, 175.3, 174.6, 170.3, 63.4, 54.7, 54.5, 50.9, 49.1, 32.3, 30.1, 29.6, 27.6, 20.8, 16.5; HRMS (ESI) m/z calculated for C₁₅H₂₈N₄O₇₊H⁺: 377.2031 [M+H]⁺; found: 377.2030.

### Compound 7

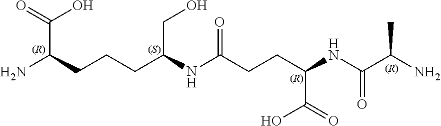

Compound 7 (42 mg, 62% over 4 steps from 5) as a white solid was prepared from N-Cbz-D-alanine as described for the preparation of 6; [α]_{D}²²=−20.7 (c=0.6, CH₃OH/H₂O=4/1); ¹H NMR (600 MHZ, D20) δ 3.99-4.07 (m, 2H), 3.78-3.79 (m, 1H), 3.62-3.65 (m, 1H), 3.48-3.51 (m, 1H), 3.39-3.42 (m, 1H), 2.21-2.25 (m, 2H), 2.00-2.02 (m, 1H), 1.71-1.88 (m, 3H), 1.50-1.51 (m, 1H), 1.46 (d, 3H J=7.1 Hz), 1.35-1.40 (m, 3H); ¹³C NMR (150 MHz, D20) δ 177.6, 175.5, 174.6, 170.0, 63.5, 54.8, 54.5, 50.9, 48.9, 32.3, 30.0, 29.6, 27.7, 20.8, 16.3; HRMS (ESI) m/z calculated for C₁₅H₂₈N₄O₇₊H⁺: 377.2031 [M+H]⁺; found: 377.2034.

### Compound 8

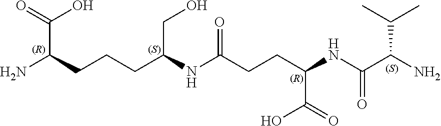

Compound 8 (42 mg, 57% over 4 steps from 5) as a white solid was prepared from N-Cbz-L-valine as described for the preparation of 6; [α]_{D}²²=8.01 (c=0.32, CH₃OH/H₂O=4/1); ¹H NMR (600 MHz, D2O) δ 4.08-4.10 (m, 1H), 3.78-3.80 (m, 1H), 3.62-3.66 (m, 2H), 3.47-3.50 (m, 1H), 3.39-3.42 (m, 1H), 2.23-2.27 (m, 2H), 1.87-2.11 (m, 2H), 1.71-1.82 (m, 3H), 1.64-1.57 (m, 1H), 1.50-1.54 (m, 3H), 0.93-0.95 (m, 6H); ¹³C NMR (150 MHZ, D20) δ 177.8, 175.2, 174.6, 169.5, 63.4, 58.9, 54.9, 54.5, 50.9, 32.4, 30.1, 29.8, 29.6, 27.5, 20.8, 17.8, 17.0; HRMS (ESI) m/z calculated for C₁₇H₃₂N₄O₇₊H⁺: 405.2344 [M+H]⁺; found: 405.2349.

### Compound 9

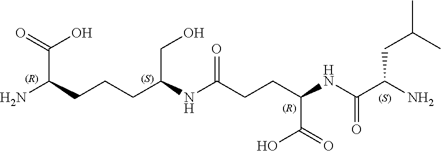

### Compound 9 (30 mg, 40% over 4 steps from 5) as a white solid was prepared

from N-Cbz-L-leucine as described for the preparation of 6; [α]_{D}²²=12.06 (c=0.46, CH₃OH/H₂O=4/1); ¹H NMR (600 MHZ, D20) δ 4.07-4.10 (m, 1H), 3.78-3.85 (m, 2H), 3.61-3.63 (m, 1H), 3.48-3.50 (m, 1H), 3.39-3.42 (m, 1H), 2.21-2.25 (m, 2H), 2.03-2.04 (m, 1H), 1.81-1.88 (m, 2H), 1.64-1.88 (m, 1H), 1.51-1.60 (m, 4H), 1.30-1.37 (m, 3H), 0.85-0.87 (m, 6H); ¹³C NMR (150 MHZ, D20) δ 177.8, 175.2, 174.6, 169.9, 63.4, 54.8, 54.5, 52.0, 50.9, 39.6, 32.4, 30.1, 29.6, 27.5, 23.9, 21.5, 21.1, 20.8; HRMS (ESI) m/z calculated for C₁₈H₃₄N₄O₇₊H⁺: 419.2500 [M+H]⁺; found: 419.2503.

### Example 2 Characterization of the Compounds of Example 1

### 2.1 NOD1-Induced NF-κB Activation

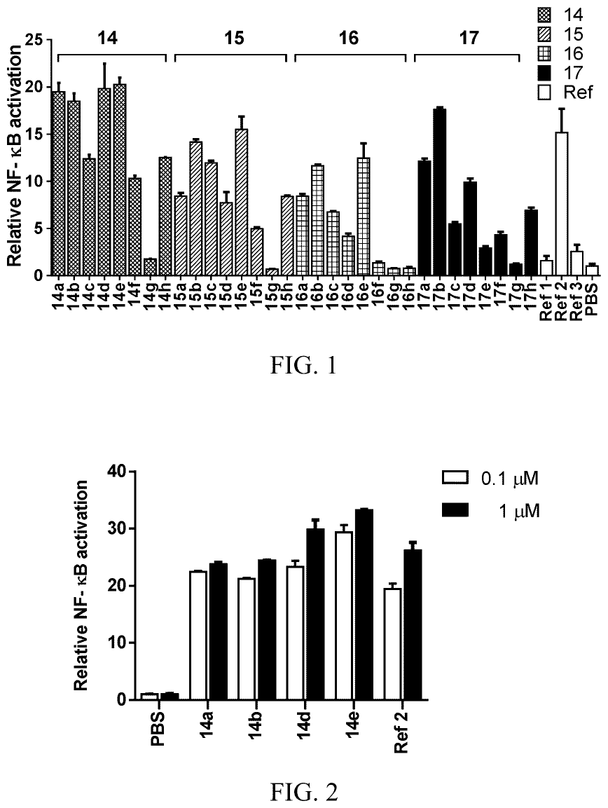

The compounds of Example 1 were evaluated for their nucleotide-binding oligomerization domain-containing protein 1 (NOD1) potential stimulation potency via use of NOD1-induced NF-κB activation assay, and results are illustrated in FIG. 1.

Tripeptides 14a, 14b, 14d, and 14e showed the highest NOD1 stimulation potency relative to the known NOD1 agonists iE-DAP, C12-iE-DAP and Tri-DAP (i.e., reference compounds 1 to 3). The result indicated that the derivatives incorporating DAHH exhibited stronger ability to stimulate NOD1 than those incorporating meso-DAP (14 vs 17). In addition, derivatives with ester moieties (series 15) were less potent than the corresponding carboxylic acids (series 14).

Further, the nature of the N-substitution on the L-Ala of the tripeptides was found to significantly affect potency. For example, 14a, bearing a C-12 moiety, and 14b, bearing a C-14 moiety, exhibited more potent stimulatory activity than 14c bearing a C-16 moiety. Notably, 14e bearing the lipophilic 11-phenoxyundecanoyl moiety on the acyl chain exhibited an attractive potency and its stimulation activity was similar to that of 14d. In contrast, when a substituent was an aromatic group, not a saturated long carbon chain, the NOD1 agonistic activity of tripeptides such as 14f, 14g, and 14h decreased dramatically, as compared to the reference compound C12-iE-DAP.

In the series 14, 14d with the 5-cyclohexylpentanoyl moiety, like a medium-chain fatty acid (MCFA), displayed a potent activity. Notably, the lipophilic moiety in 14d is not commonly expected; presumably, the bulky cyclohexyl moiety might contribute more hydrophilic interactions with residues in NOD1, but more detailed studies remain to be explored in the future.

Taken together, these results emphasized the importance of N-acyl substitution for beneficial NOD1 agonistic activity, and established chiral DAHH as a valid fragment for the development of NOD1 agonists. Importantly, it was found that not only the N-substituted A-iE-DAHH-based tripeptide 14 was a promising scaffold, but also several analogues such as 14a, 14b, 14d, and 14e exhibited a high NOD1 stimulation activity compared to known reference NOD1 agonists.

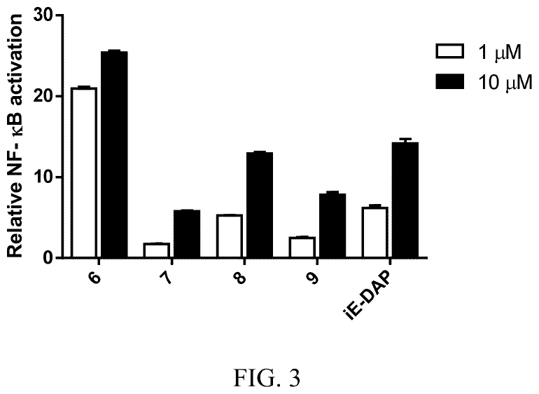

Subsequent comparison of selected 6, 7, 8, 9, 14a, 14b, 14d, and 14e in the NOD1 stimulation study were tested at two concentrations (0.1 μM vs 1 μM), and results are depicted in FIGS. 2 and 3.

As depicted, 14d and 14e at 1 μM concentration exhibited higher activity with 30-and 33-fold increase of NF-κB activity, respectively, compared to 14a, 14b, and the reference compound 2 (FIG. 2). Similarly, compound 6 at 1 μM concentration exhibited higher activity with 20-fold increase of NF-κB activity, as compared to the iE-DAP (i.e., the reference compound 1) (FIG. 3). Thus, potent NOD1 agonists 14d and 14e were chosen for the subsequent in vivo study of their adjuvant activity.

### 2.2 The Adjuvant Activity of 6, 14d and 14e

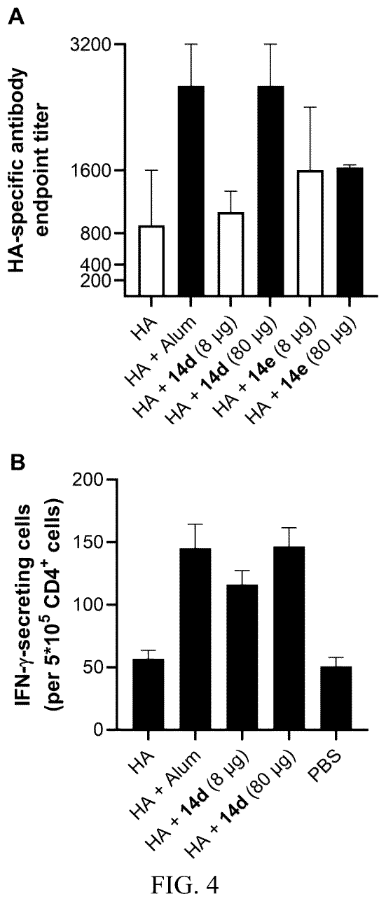

In this example, influenza hemagglutinin (HA) was used as the antigen to immunize mice and aluminum hydroxide (Alum), a conventional adjuvant, was applied as a control adjuvant. As shown in FIG. 4A, the addition of 14d, but not 14e, increased the amount of the antigen-specific antibodies. The antibody increase caused by 14d was comparable to the increase resulted from the addition of Alum.

Whether the adjuvant could increase antigen-specific T-cell response was further evaluated by ELISpot assay, which offers multi-dimensional, quantitative assessment of effector function(s) at the single cell level. The splenocytes from immunized mice were collected and secreting cytokines IFN-γ from individual responding cells were monitored. The results confirmed that 14d could indeed increase IFN-γ-secreting cells (FIG. 4B), suggesting that HA-specific T-cell function, specifically Th1 response, could be promoted by 14d.

This proof-of-concept study demonstrated the new DAHH containing tripeptide 14d may induce innate immune responses through NOD1 stimulatory activity and its immunomodulatory activity exhibited an adjuvant effect to boost the level of antigen-specific antibodies.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of the present disclosure.

1. A compound of formula (I), a pharmaceutically acceptable salt, solvate or stereoisomer thereof, 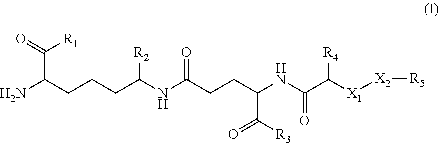
wherein,
X₁ and X₂ are independently nil, —NH or —(C═O);
R₁ and R₃ are independently hydroxyl or alkoxy;
R₂ is —COOH or —CH₂OH;
R₄ is hydrogen or C₁₋₆ alkyl;
R₅ is hydrogen or C₁₋₂₀ alkyl optionally substituted with a substituent selected from the group consisting of cycloalkyl, phenyl, biphenyl and —O-phenyl; and
the phenyl, the biphenyl or the —O-phenyl is optionally substituted with one or more halo, alkyl, or alkoxy.

2. The compound of claim 1, wherein X₁ is nil, X₂ is —NH, R₁ is hydroxy, R₂ is methoxy, R₃ is hydroxy, R₄ is isopropyl or isobutyl, and R₅ is hydrogen.

3. The compound of claim 2, wherein the compound is selected from the

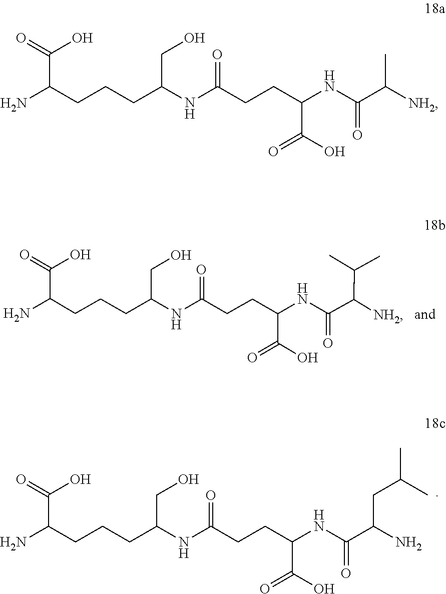 [SMILES: CC(N)C(=O)NC(CCC(=O)NC(CO)CCCC(N)C(=O)O)C(=O)O ; CC(C)C(N)C(=O)NC(CCC(=O)NC(CO)CCCC(N)C(=O)O)C(=O)O ; CC(C)CC(N)C(=O)NC(CCC(=O)NC(CO)CCCC(N)C(=O)O)C(=O)O]

4. The compound of claim 1, wherein the compound has the structure of formula (II), 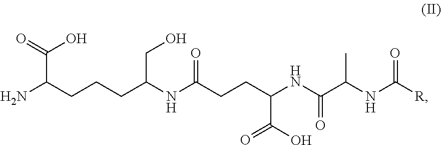
wherein,
R is C₁₋₂₀ alkyl optionally substituted with a substituent selected from the group consisting of cycloalkyl, phenyl, biphenyl and —O-phenyl; and
the phenyl, the biphenyl or the —O-phenyl is optionally substituted with one or more halo, alkyl, or alkoxyl.

5. The compound of claim 4, wherein the compound is selected from the

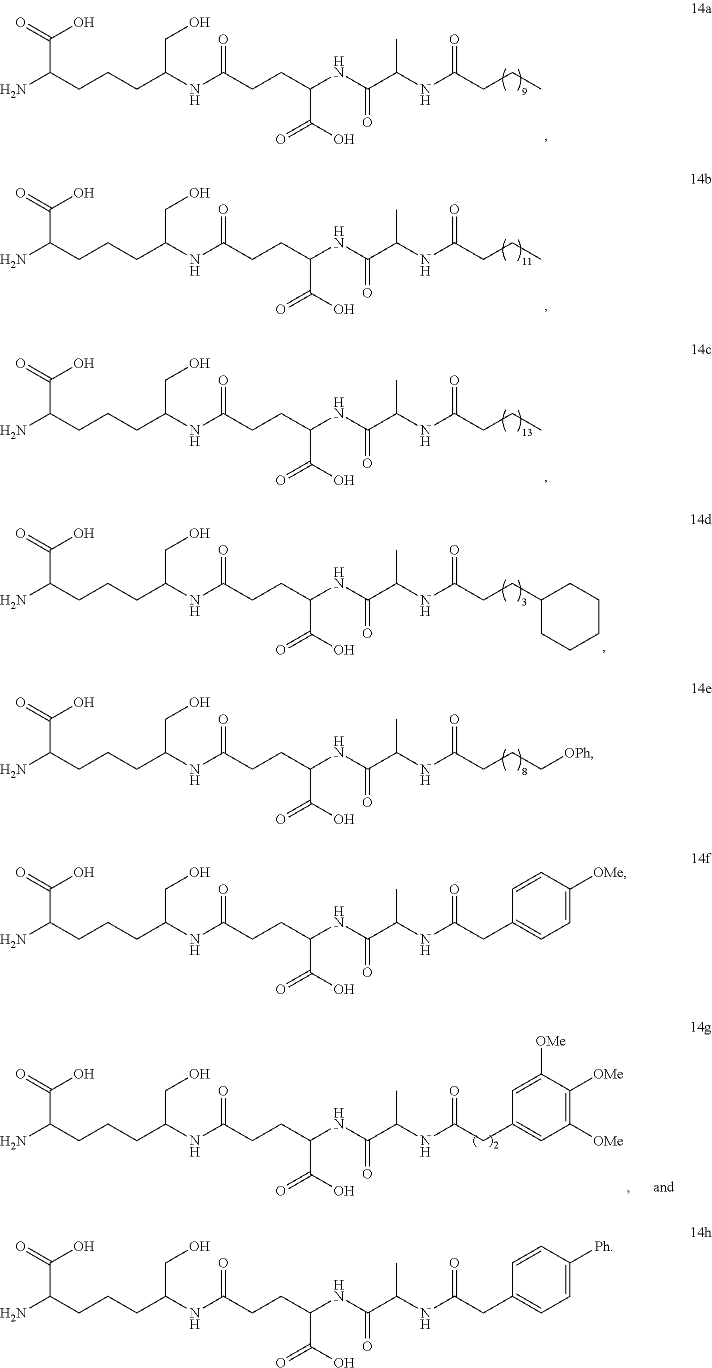

6. The compound of claim 5, wherein the compound is

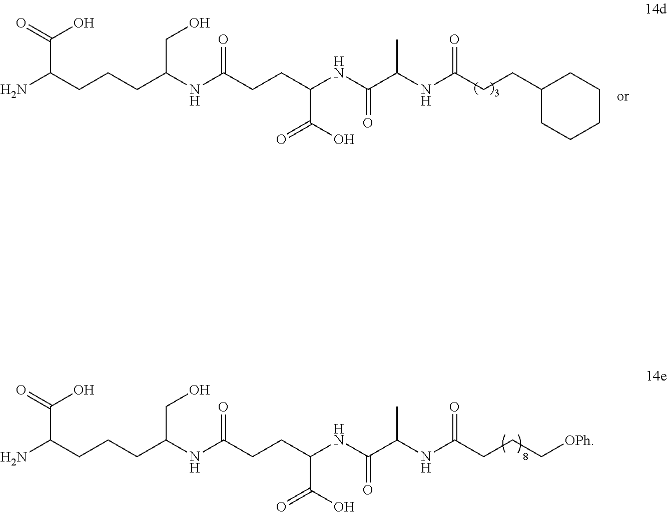

7. The compound of claim 1, wherein the compound has the structure of formula (III), 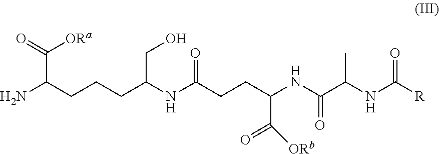
wherein,
R^{a} and R^{b} are independently C₁₋₆alkyl;
R is C₁₋₂₀ alkyl optionally substituted with a substituent selected from the group consisting of cycloalkyl, phenyl, biphenyl and —O-phenyl; and
the phenyl, the biphenyl or the —O-phenyl is optionally substituted with one or more halo, alkyl, or alkoxyl.

8. The compound of claim 7, wherein the compound is selected from the

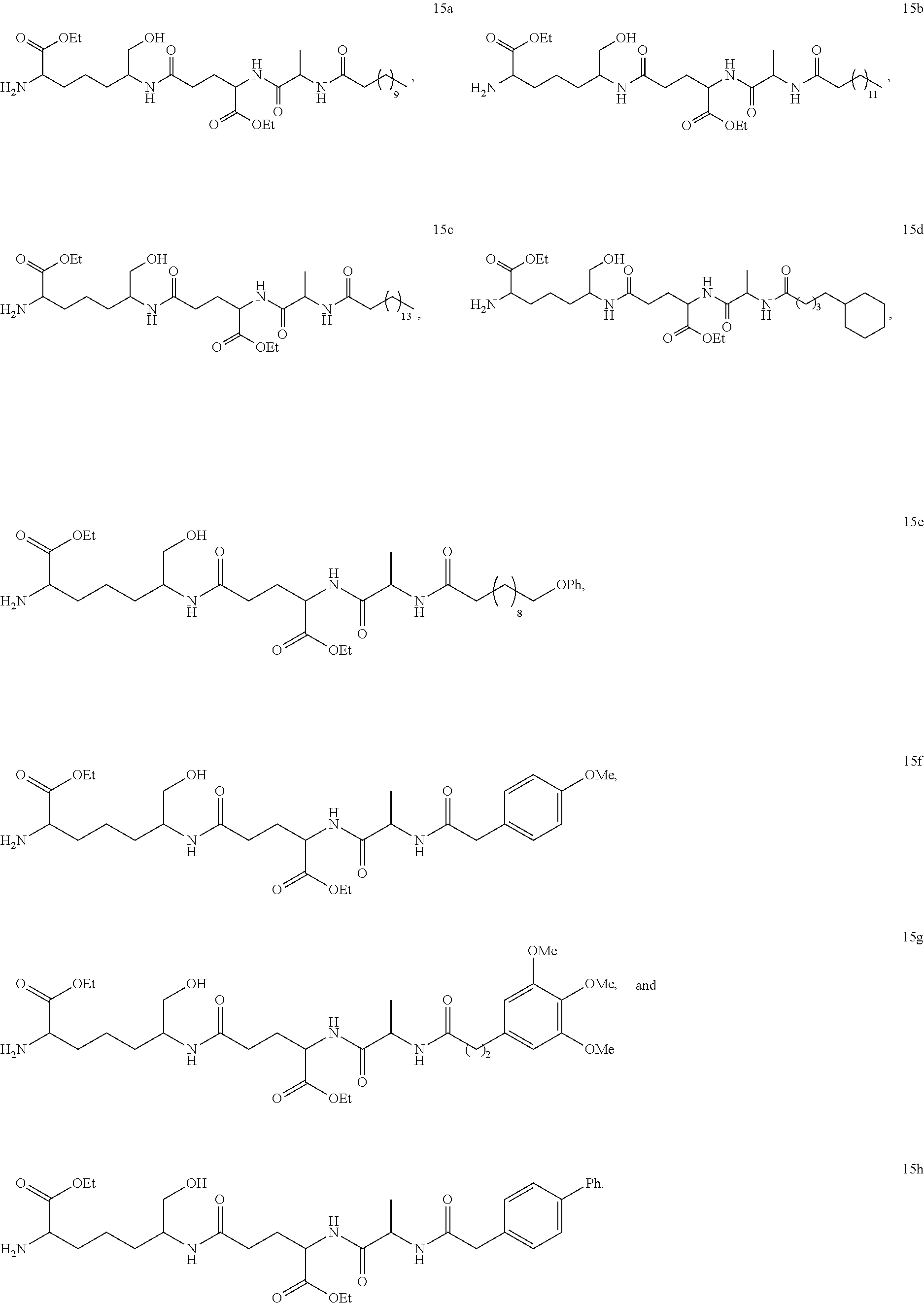

9. The compound of claim 1, wherein the compound has the structure of formula (IV), 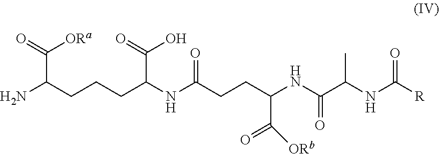
wherein,
R^{a} and R^{b} are independently C₁₋₆ alkyl;
R is C₁₋₂₀ alkyl optionally substituted with a substituent selected from the group consisting of cycloalkyl, phenyl, biphenyl and —O-phenyl; and
the phenyl, the biphenyl or the —O-phenyl is optionally substituted with one or more halo, alkyl, or alkoxyl.

10. The compound of claim 9, wherein the compound is selected from the

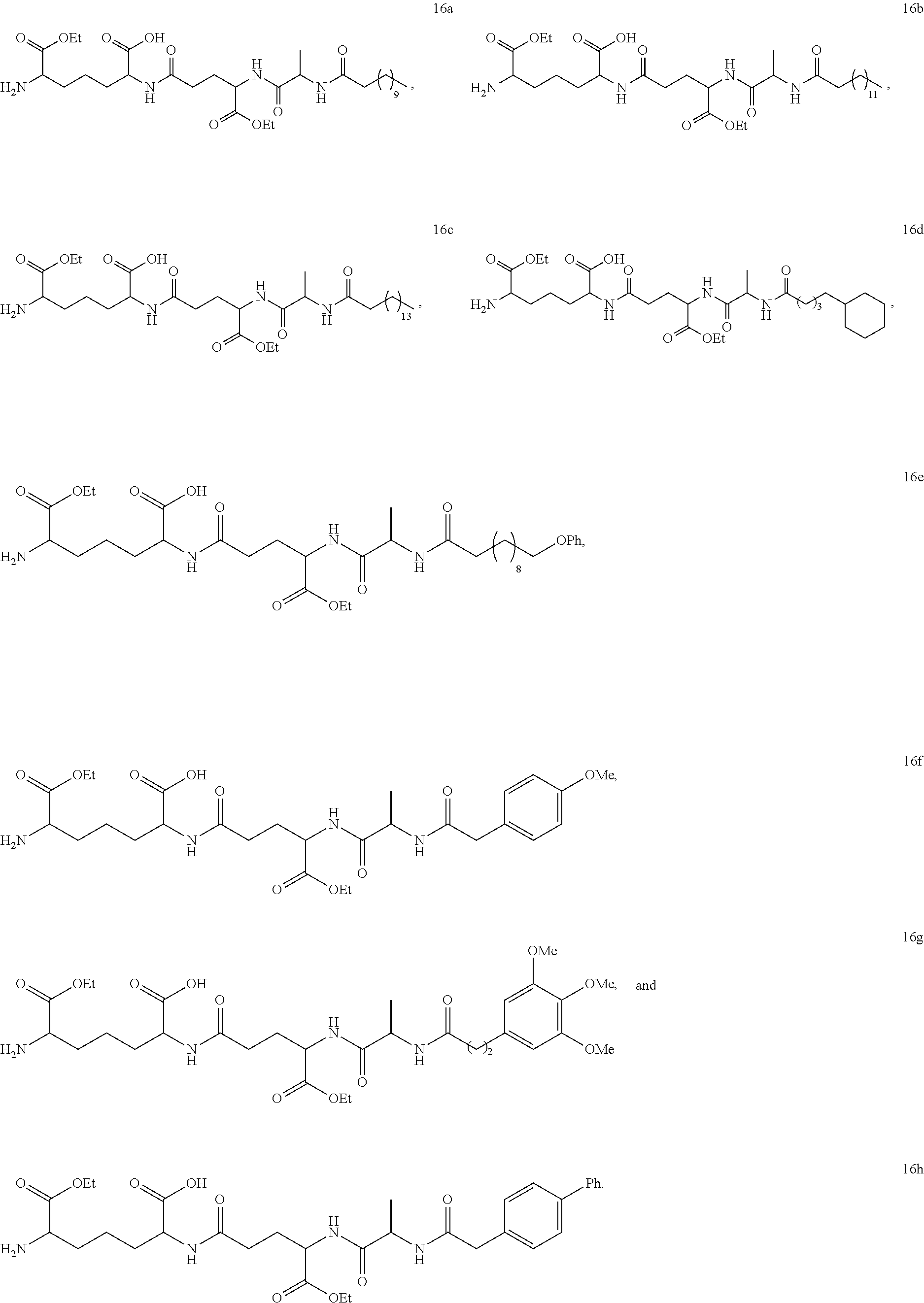

11. The compound of claim 1, wherein the compound has the structure of formula (V) 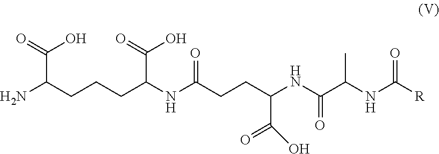
wherein,
R is C₁₋₂₀ alkyl optionally substituted with a substituent selected from the group consisting of cycloalkyl, phenyl, biphenyl and —O-phenyl; and
the phenyl, the biphenyl or the —O-phenyl is optionally substituted with one or more halo, alkyl, or alkoxyl.

12. The compound of claim 11, wherein the compound is selected from the

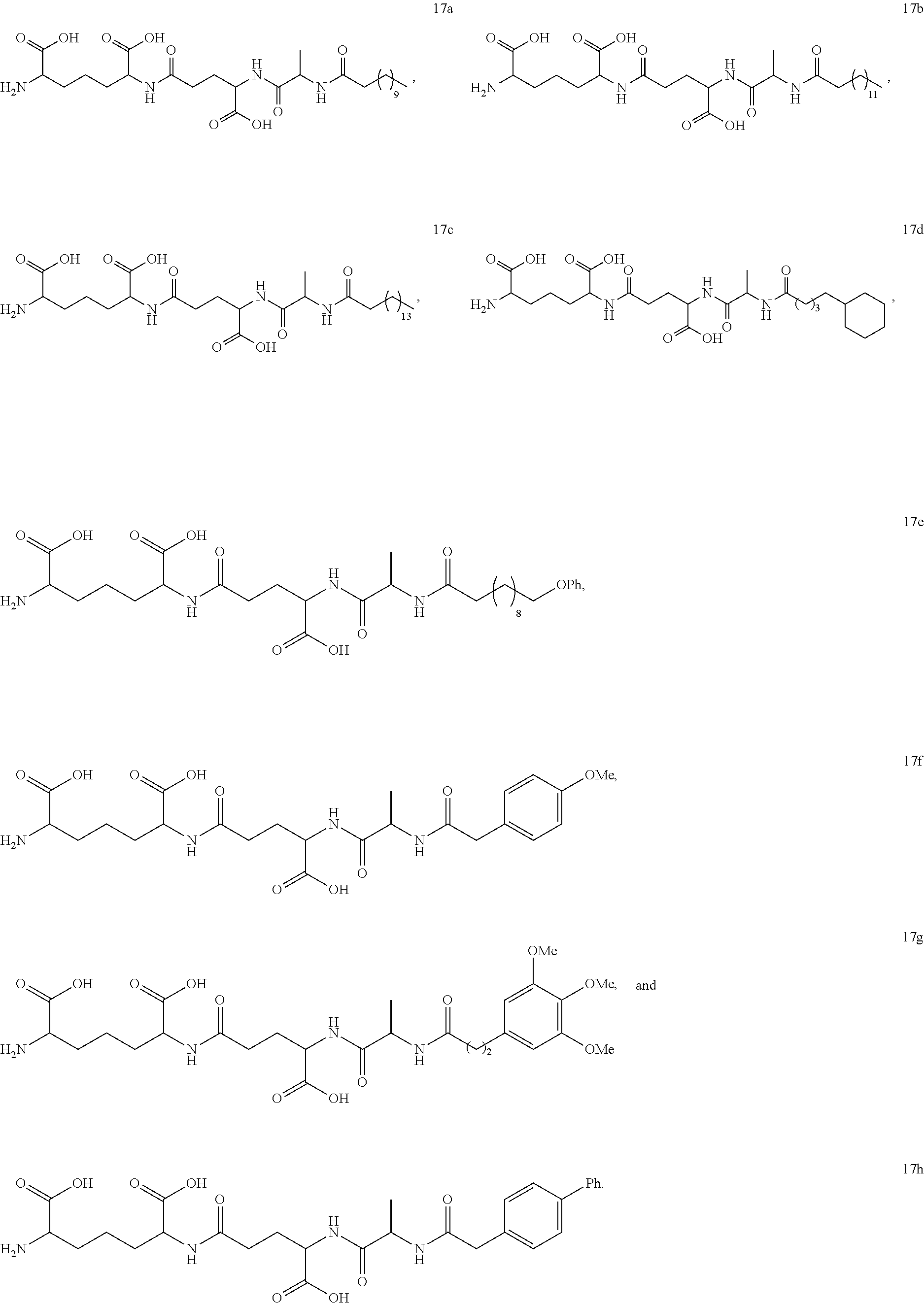

13. The compound of claim 1, wherein the compound has the structure of formula (VI) 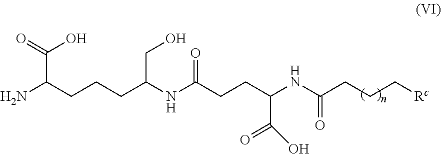
wherein,
n is an integral between 3 to 10;
R^{c} is H, cycloalkyl, phenyl, biphenyl or —O-phenyl; and
the phenyl, the biphenyl or the —O-phenyl is optionally substituted with one or more halo, alkyl, or alkoxyl.

14. The compound of claim 13, wherein the compound is selected from the

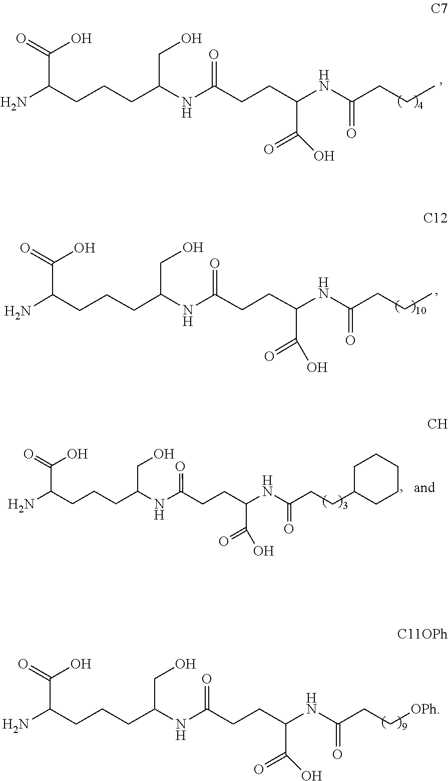

15. A vaccine comprising an immunogen and the compound of claim 1, the pharmaceutically acceptable salt, solvate or stereoisomer thereof, wherein the immunogen is capable of eliciting an immune response against a respiratory virus, and the compound of claim 1, the pharmaceutically acceptable salt, solvate or stereoisomer thereof is capable of enhancing the immune response elicited by the immunogen.

16. The vaccine of claim 15, wherein the immunogen is an antigen selected from the group consisting of a subunit antigen, an inactivated whole virus, a live attenuated virus, and a combination thereof.

17. The vaccine of claim 16, wherein the compound is

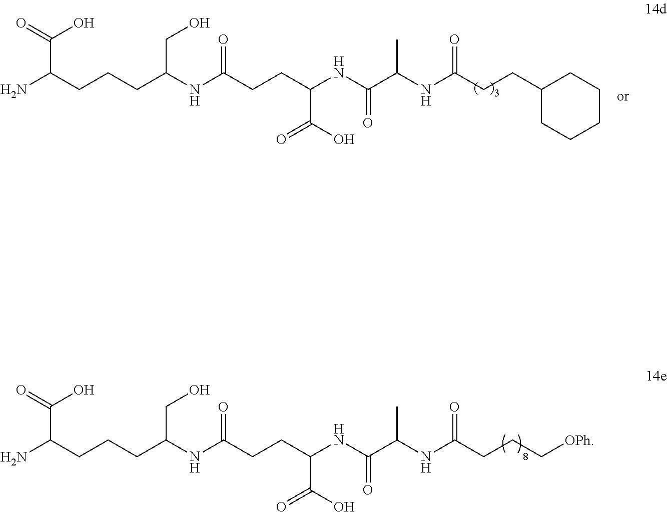

18. The vaccine of claim 16, wherein the respiratory virus is an influenza virus.

19. The vaccine of claim 18, wherein the influenza virus is an avian influenza virus or a seasonal influenza virus.

20. The vaccine of claim 19, wherein the avian influenza virus is H₅N1 or H₇N9.

21. A method of immunizing a subject against a viral respiratory infection comprising administering to the subject an effective amount of the vaccine of claim 15.

22. The method of claim 21, wherein the compound is

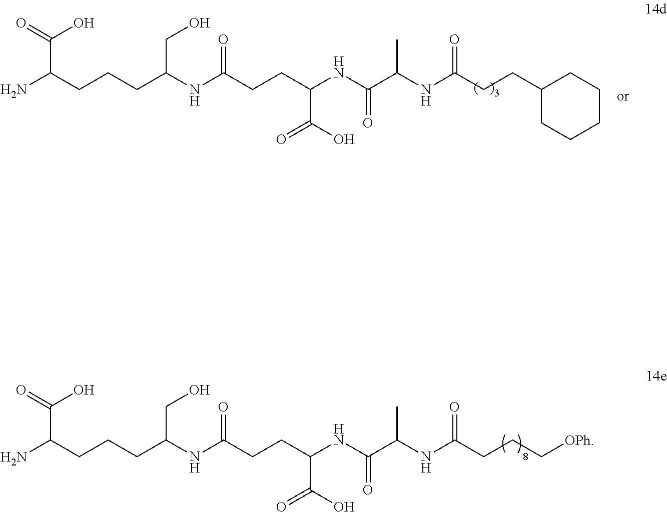

23. The method of claim 21, wherein the immunogen is an antigen selected from the group consisting of a subunit antigen, an inactivated whole virus, a live attenuated virus, and a combination thereof.

24. The method of claim 23, wherein the respiratory virus is an influenza virus.

25. The method of claim 24, wherein the influenza virus is an avian influenza virus or a seasonal influenza virus.

26. The method of claim 25, wherein the avian influenza virus is H₅N1 or H₇N9.

27. The method of claim 21, wherein the subject is a human.

28-30. (canceled)
